(19) United States
(12) Patent Application Publication
(10) Pub. No.: US 2013/0040837 A1
(43) Pub. Date: Feb. 14, 2013

(54) APTAMER CELL COMPOSITIONS

(71) Applicants: Jeffrey M. Karp, Brookline, MA (US); Wei Li Loh, Singapore (SG); Debanjan Sarkar, Williamsville, NY (US); Sebastian Schaefer, Berlin (DE); Weian Zhao, Irvine, CA (US)

(72) Inventors: Jeffrey M. Karp, Brookline, MA (US); Wei Li Loh, Singapore (SG); Debanjan Sarkar, Williamsville, NY (US); Sebastian Schaefer, Berlin (DE); Weian Zhao, Irvine, CA (US)

(73) Assignee: BRIGHAM AND WOMEN'S HOSPITAL, INC., Boston, MA (US)

(21) Appl. No.: 13/513,401

(22) PCT Filed: Dec. 1, 2010
(86) PCT No.: PCT/US10/58613

§ 371 Date: Oct. 26, 2012

Related U.S. Application Data
(60) Provisional application No. 61/265,387, filed on Dec. 1, 2009.
(60) Provisional application No. 61/418,778, filed on Dec. 1, 2010.

(51) Int. Cl. C12Q 1/68; C40B 30/04; C12N 5/07

(52) U.S. Cl. 506/9; 435/325; 435/6.1; 435/6.12

(57) ABSTRACT

A composition includes an isolated cell, wherein a surface of the cell is attached to a nucleic acid that specifically binds to a non-nucleic target.

### CLAIM OF PRIORITY

This application claims priority to U.S. Patent Application Ser. No. 61/265,387, filed on Dec. 1, 2009, and U.S. Patent Application Ser. No. 61/418,778, filed on Dec. 1, 2010. The entire contents of both prior applications are incorporated herein by reference.

### TECHNICAL FIELD

This invention relates to compositions for detection of molecules and targeting of cells.

### BACKGROUND

The local nanoenvironment surrounding the cell membrane impacts cells function. In particular, cells respond to cytokines and growth factors that surround them.

Intercellular signaling is divided into endocrine, paracrine, autocrine, and juxtacrine signaling. Endocrine signals are produced by endocrine cells and travel through the blood to reach all parts of the body. Paracrine signals target only cells in the vicinity of the emitting cell (e.g., neurotransmitters). Autocrine signals affect only cells that are of the same cell type as the emitting cell (e.g., immune cells). Juxtacrine signals are transmitted along cell membranes via protein or lipid components integral to the membrane and are capable of affecting either the emitting cell or cells immediately adjacent.

Some signaling molecules degrade very quickly or are taken up quickly. In these cases monitoring of these signals with traditional technology is not practically feasible. For instance, the communication between endothelial cells (ECs) (or cancer cells) with mesenchymal stem cells (MSCs), mainly via growth factors such as vascular endothelial growth factor (VEGF), platelet-derived growth factor (PDGF), is highly implicated in angiogenesis, tumor growth, etc. Cancer cells attract MSCs through released PDGF, among other factors to tumor sites, particularly to tumor vessels, suggesting a supportive role in angiogenesis (Beckermann et al., 2008, Br. J. Cancer, 99:622-631). In another example, PDGF secreted by chronic lymphocytic leukemic B-cells is capable of regulating the activation and function of MSC which demonstrated implications for leukemic cell/stromal cell crosstalk (Ding, W. 50th ASH Annual Meeting and Exposition, San Francisco, Calif., USA, 2008, December 6-9).

However, the signals controlling cell-cell communications are poorly understood. For instance, little is known about the factors that enable the mobilization of MSC from the bone marrow into the blood stream and their recruitment to and retention in the tumor (Beckermann et al., 2008, Br. J. Cancer, 99:622-631).

The study of such local nanoenvironment and specifically how cells sense the molecules in such a niche is therefore crucial, and the knowledge obtained from these studies can, in turn, serve as guide for developing better therapies for the treatment of certain diseases. However, most of cell signaling processes are poorly understood, and more importantly there are no ideal tools to study such processes in a real-time in situ. Traditional techniques such as reverse transcriptase polymerase chain reaction (RT-PCR), flow cytometry, and immunofluorescence microscopy often require stepwise staining and multiple manipulations before analysis and are typically not capable of real-time in situ monitoring. Enzyme-linked immunosorbent assays (ELISA) are mainly used to characterize the cytokine concentrations in bulk medium and do not provide detailed information regarding the area within a 0-1000 nm range of the cell surface. Fluorescent dyes, nanoparticles such as quantum dots and iron oxide particles, often conjugated with antibodies, have been applied to stain cells followed by flow cytometry, fluorescent microscopy and magnetic resonance imaging (MRI), respectively. These give an overall marker expression on the cell membrane, but still do not provide information on the markers coming to the cell membrane in real time.

### SUMMARY

This application discloses the immobilization of aptamers on cell membranes. In the case of aptamer modified cell systems for sensor applications, sensors on the cell surface enable the study of the local nanoenvironment of cells and cell-cell communications and signaling. These systems are useful to study how cells respond to a stimulus in vitro and in vivo. Aptamers immobilized on the surface of cells can also promote desirable cell-cell interactions. Long DNA probes containing aptamers can also be used for ultrasensitive detection of markers in biological solutions or on cell membranes. This disclosure enables ultra-sensitive rapid detection of biological markers for use in drug screening (e.g., this can significantly reduce the number of cells and/or time required for toxicity screens, which is pertinent for cell types such as hepatocytes that are difficult to culture).

In one aspect, the disclosure features compositions that include an isolated cell (e.g., a stem cell, progenitor cell, reprogrammed cell, differentiated cell, blood cell, or platelet), wherein a nucleic acid (e.g., an aptamer) that specifically binds to a non-nucleic acid target is attached to the surface of a cell. The nucleic acid can be immobilized on the cell surface either covalently or non-covalently. In some embodiments, a connector moiety is present between the cell and the nucleic acid, and the connector moiety as well can be attached either covalently or non-covalently to each of the cell and the nucleic acid. In some embodiments, the connector moiety contains biotin and/or poly(ethylene glycol). In some embodiments, the nucleic acid includes several (e.g., more than 10, 20, 50, 100, 200, 500, or 1000) target binding sequences and can optionally include one or more catalytic nucleic acid sequences.

In some embodiments, the nucleic acid includes two or more polynucleotide strands. For example, the nucleic acid can include an aptamer strand and another strand complementary to at least a portion of the aptamer strand. In another example, each of the two or more polynucleotide strands are aptamers that bind to the same or different targets. When fluorescent dyes and/or quenchers are used, each strand of the two or more polynucleotide strands can include one or more fluorescent dyes or quenchers. In some embodiments, the sensitivity of the sensor can be modified by adjusting the length of base pairs in the complementary domain of the sensor when it folds.

In some embodiments, the nucleic acid is modified with one or more sensor moieties that enable detection of binding to the non-nucleic acid target. Non-limiting examples of sensor moieties include fluorescent dyes (e.g., FITC, FAM, Alexa 488, TAMRA, Cy3, Cy5, Cy5.5) and fluorescence quenchers (e.g., dabcyl). Binding of the nucleic acid to the target can result in modification (e.g., increase or decrease) of a fluorescent signal (e.g., a fluorescence resonance energy transfer (FRET) signal). When two sensor moieties are present, a detection event can result in an increase of the intensity of one signal and a decrease in the intensity of a second signal. In some embodiments, a fluorescent signal is modified (e.g., increased or decreased) based on a conformational change of the nucleic acid on binding to the target. In some embodiments, the nucleic acid is modified to enhance nuclease resistance (e.g., with PEG or an inverted nucleotide cap).

In some embodiments, the compositions are capable of real time monitoring of a biological event. In some embodiments, the compositions are capable of detecting molecules present locally (e.g., within 0-1000 nm) of a membrane of the cell.

In some embodiments, the nucleic acid is engineered to function under physiological conditions, e.g., in the presence of divalent metal ions (e.g., Mg²⁺, Ca²⁺). Further, the disclosure features methods of modifying a nucleic acid that binds to a non-nucleic acid target (e.g., an aptamer) by reducing the length of an annealed region of the nucleic acid created on binding of the nucleic acid to the target. These methods can result in increased function of the nucleic acid under physiological conditions, e.g., in the presence of divalent metal ions (e.g., Mg²⁺, Ca²⁺).

This disclosure also features methods of detecting target molecules using the compositions described herein. In some embodiments, the compositions include mesenchymal stem cells and are used to detect PDGF. In some embodiments, the sensors are used to detect molecules released from the same cells upon which the nucleic acid is immobilized. The methods can include contacting a composition described herein with a sample (e.g., a biological sample) suspected of containing the target molecule and assaying binding of the composition to a target molecule in the sample. In some embodiments, assaying binding of the composition can involve flow cytometry and/or microscopy (e.g., to detect a fluorescent signal).

In some embodiments, the compositions described herein can include a nucleic acid that can bind specifically to a cell surface antigen, e.g., a selectin (e.g., L-, P- or E-selectin), or an extracellular matrix protein. Such compositions can be used to promote cell-cell interactions, e.g., binding under dynamic flow conditions or cell adhesion (e.g., cell rolling and/or firm adhesion).

This disclosure also features methods of targeting the compositions described herein to specific locations (e.g., a surface, cell, or tissue). The methods can include bringing the composition into contact with the location, wherein the location includes a target of the nucleic acid.

This disclosure also features compositions that include a particle (e.g., a bead, nanoparticle, or microparticle) attached to a nucleic acid (e.g., an aptamer) that specifically binds to a non-nucleic acid target. In some embodiments, the nucleic acid includes several (e.g., more than 10, 20, 50, 100, 200, 500, or 1000) target binding sequences (e.g., aptamers) and can optionally include one or more catalytic nucleic acid sequences (e.g., that convert chromogenic and/or fluorogenic dyes to color and/or fluorescent signals). Also featured are methods of using such compositions to detect the targets (e.g., in vivo).

The disclosure also features nucleic acid probes for detection of targets in biological solutions and/or on cell membranes. In some embodiments, the nucleic acid probes include several (e.g., more than 10, 20, 50, 100, 200, 500, or 1000) target binding sequences (e.g., aptamers) and can optionally include one or more catalytic nucleic acid sequences. In some embodiments, the nucleic acid probes are made by rolling circle amplification (RCA) of a template that includes one or more target binding sequences and one or more catalytic nucleic acid sequences (e.g., that convert chromogenic and/or fluorogenic dyes to color and/or fluorescent signals). In some embodiments, the probes allow for ultrasensitive detection of the target in solution (e.g., at femtomolar, picomolar, or nanomolar concentrations) or on a cell membrane (e.g., at less than 100 targets, less than 80 targets, less than 60 targets, less than 40 targets, less than 20 targets, less than 10 targets, less than 5 targets, or a single target per cell). The probes can be attached to a solid substrate (e.g., glass, gold, plastic (e.g. poly(styrene)), silicon) or to a cell membrane. In some embodiments, the nucleic acid probes include one or more fluorescent moieties (e.g., one or more different types of fluorescent moieties). In some embodiments, the probes can be used to detect molecules relevant to cell toxicity.

In some embodiments of the above compositions, the nucleic acids can be internalized by the cell to detect intracellular biological markers, e.g., in a compartment of the cell (e.g., a lysosome, cytoplasm, etc.).

The disclosure also features methods of detecting targets in solution using an Enzyme-linked Aptamer Sorbent Assay (ELASA). The methods can include contacting a capture agent (e.g., a nucleic acid (e.g., an aptamer) that specifically binds to a non-nucleic acid target) bound on a solid support with a solution (e.g., a biological solution) such that a target of the nucleic acid binds to the nucleic acid, contacting the target bound to the nucleic acid with a second nucleic acid (e.g., an aptamer) that specifically binds to the non-nucleic acid target, wherein the second nucleic acid include (e.g., is covalently or noncovalently attached to) an RCA primer; contacting the RCA primer with an RCA template, performing an RCA reaction using the RCA primer and RCA template, and detecting a product of the RCA reaction. In some embodiments, the RCA template encodes a catalytic nucleic acid. In such cases, detection of the product of the RCA reaction can include detecting a product of the reaction stimulated by the catalytic nucleic acid, e.g., a colored and/or fluorescent signal.

The aptamer-engineered cells disclosed herein can be used in a variety of applications including: 1) multiplex, high throughput analysis of cell-cell interactions and drug screening, 2) real-time and in situ study of the cellular nanoenvironment, 3) analyzing how cells respond to a specific stimulus, 4) studying cell niche in vivo, 5) observing in vivo cell behaviors including trafficking, homing and differentiation, 6) multiplex, high throughput and ultrasensitive detection of cytokines or growth factors in the cell culture medium, 7) facile and ultrasensitive detection of cell surface markers, 8) cell targeting and cell therapy, and 9) promotion of desirable cell-cell interactions.

In another aspect, the disclosure features a composition including a sensor immobilized on the surface of a cell that provides two signals in the presence of a stimulus enabling an enhanced level of detection.

In another aspect, the disclosure features compositions that include nucleic acids that are produced on a substrate using rolling circle amplification (RCA) to capture and detect cells. In some embodiments, an RCA primer is attached to the substrate covalently or noncovalently. In some embodiments, an RCA circular template is annealed with the primer before or after immobilization of the primer to the substrates. In some embodiments, the nucleic acids contain a plurality of aptamers (e.g., the same or different aptamers). The aptamers can bind to antigens on cells, e.g., cancer cells (e.g., circulating tumor cells). The substrate can include one or more of glass, silicon, gold, polymer and plastic. In some embodiments, the substrates are integrated in a microfluidic device.

In another aspect, the disclosure features a device immobilized on a cell surface, wherein the device is capable of measuring a biological event and converts the event into a detectable signal, e.g., that can be read by an observer or by an instrument. The immobilization can be achieved, e.g., by one or more of chemical and physical means. In some embodiments, the device includes at least one binding domain that binds to at least one cell non-specifically, specifically or non-specifically and specifically. In some embodiments, the immobilization involves an initial transport step through which the device reaches the cell surface from the extracellular environment. In some embodiments, the immobilization is achieved in fewer than 5 minutes. In some embodiments, the concentration of the device on the cell surface is modifiable by altering concentration of the device in the extracellular environment. Optionally, the device is not a product of gene modification. The biological event to be measured can reflect a biological pathway or consequence, e.g., the presence of a biological moiety that is to be detected. The biological moiety can be released from inside the cell to the extracellular environment and/or transported from the extracellular environment to the cell surface. In some embodiments, the moiety is from the same cell to which the device is immobilized or the moiety is from at least one different cell. In some embodiments, the moiety is released upon stimulation of the cell (e.g., via cell-cell communication).

In some aspects of the above compositions, a nucleic acid that binds to a target molecule can be substituted with another sensor moiety capable of real-time detection by generating a signal in the presence of a target (e.g., an enzyme, sugar, protein, etc.) or a condition (e.g., pH). In some embodiments, the sensor moiety is a polymer. Exemplary polymeric sensors are described in Osada and De Rossi, eds., Polymer Sensors and Actuators, Springer, 1999. In some embodiments, the sensor is a peptide that can be cleaved in the presence of an enzymatic target. The peptide can incorporate one or more fluorescent and/or quenching moieties such that a signal can be detected on cleavage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic diagram showing immobilization and functional components for compositions disclosed herein.

FIGS. 2A-2C are schematic diagrams showing uses of compositions disclosed herein for monitoring of the cell nanoenvironment and cell-cell signaling (2A), detection of markers in solutions and on cell membranes (2B), and promoting cell-substrate or cell-cell interactions (2C).

FIG. 3 is a schematic diagram showing the detection regions of the aptamers on cells as disclosed herein compared to those in traditional ELISA, protein array, and immunostaining assays.

FIG. 4 depicts a covalent conjugation strategy for attaching biotin modified aptamers on cells.

FIGS. 5A-5B depict an exemplary strategy for modification of a PDGF aptamer (5A; SEQ ID NO:1) to be cell surface adaptable (5B). The single stranded PDGF aptamer is extended at one end with a short oligonucleotide that can hybridize with its complementary oligonucleotide strand. Two dyes and an anchor moiety (e.g., biotin or a lipid) can be easily accommodated on these two separated strands during synthesis which are then annealed together before attaching to cells.

FIGS. 6A-6B depict an exemplary strategy for modification of a PDGF aptamer (6A; SEQ ID NO:1) for increased function in the presence of divalent metal ions (6B; SEQ ID NO:2).

FIGS. 7A-7C depict an exemplary strategy for optimizing FRET signal of a PDGF aptamer (7A; SEQ ID NO:1) by tuning the dye positions on the sensor strands (7B, 7C).

FIGS. 8A-8B depict exemplary aptamer constructs on cells. In 8A, one of the two nucleic acid strands is an aptamer strand which includes an aptamer domain that binds specifically to the target and an extended oligonucleotide. The second strand in the sensor construct is complementary to and therefore binds to the extended domain in the aptamer strand. Dyes and anchor molecules can be accommodated onto these two strands which enable both sensing functions and being adaptable on cell surface. In 8B, both stands are aptamers. This will be suitable for target molecules that have two binding sites to aptamers. In both cases, the binding of target places the two dyes into close proximity which gives a detectable signal, e.g., via fluorescence quenching or FRET mechanisms.

FIGS. 9A-9D depict methods of using aptamer sensor on cells to detect molecules at the cell surface nanoenvironment. 9A, Target molecules are detected in a medium. 9B, target molecules released from a second cell are detected. 9C, target molecules released from the same cell are detected. 9D, multiple aptamer sensors are attached to the same or different cells for monitoring of multiple target molecules and/or multiple biological processes simultaneously.

FIGS. 10A-10C depict exemplary experimental designs and microfluidic devices for the use of aptamer sensors on cells to detect molecules in cell nanoenvironment. In 10A, aptamer sensors on cells detect added target molecules. PDGF is infused via a nanochannel of microfluidic device. The aptamer sensors modified MSCs on the other side of channel will response to PDGF differently depending on the diffuse concentration profile of PDGF. For example, the cells that are surrounded with higher PDGF concentration give higher signal. In 10B, aptamer sensors on cells detect the target molecules released by the same cell. Thrombin is infused via a nanochannel or microfluidic device to activate ECs or platelets to release PDGF. The aptamer sensors on the cells then detect the released PDGF. The response can also be dependent on trigger molecule, e.g., thrombin, concentration. In 10C, aptamer sensors on cells detect target molecules released from a different cell. PDGF is released from ECs (or platelets) upon thrombin activation. The aptamer sensor modified MSCs will respond to PDGF in a concentration dependent manner, e.g., based on the distance between the MSC and ECs (or platelets).

FIGS. 11A-11B are schematic diagrams of PDGF aptamers on beads. 11A, The FAM is labeled at 5′ end of aptamer (SEQ ID NO:3) and a quencher, dabcyl, is labeled at the 3′ end of the complementary strand (SEQ ID NO:4), on which a biotin molecule is attached at the other end. Upon binding to the target PDGF, aptamer undergoes a conformational change which brings FAM and dabcyl closer to each other and therefore the fluorescence of FAM is quenched. 11B, One base pair C-G on aptamer sensor (SEQ ID NO:5) is eliminated by changing a C base to A base, and the complementary strand (SEQ ID NO:4) is unchanged. Sensor 2 has less nonspecific folding and better performance in the presence of divalent metal ions than sensor 1.

FIGS. 11C-11D are bar graphs depicting the performance of sensors 1 and 2 immobilized on streptavidin beads for detection of PDGF in PBS (11C) and PBS with 0.9 mM CaCl₂ and 0.5 mM MgCl₂ (11D) were studied by flow cytometry. Upon adding PDGF (10 nM), the FAM green signal is quenched. Sensor 2 functions better than sensor 1 in PBS with and without Ca/Mg.

FIGS. 12A-12B are fluorescence micrographs depicting fluorescence quenching of sensor 2 on streptavidin beads before (12A) and after (12B) addition of 10 nM PDGF.

FIG. 13A is a schematic diagram of a PDGF aptamer on a bead. The 5′ end of the aptamer (SEQ ID NO:3) was labeled with FAM and the complementary strand (SEQ ID NO:4) was labeled with TAMRA, for use as FRET donor dye and acceptor dye, respectively. Upon adding PDGF, the aptamer folds and brings two dyes into close proximity where FAM fluorescence signal is quenched by TAMRA.

FIG. 13B is a graph depicting the amount of fluorescence quenching of the PDGF aptamer in 13A following addition of PDGF at various concentrations, analyzed by flow cytometry and plotted as signal in the Y axis.

FIGS. 14A-14C are bar graphs depicting quenching performance of sensor 2 on MSCs in PBS (14A), PBS with 0.9 mM CaCl₂ and 0.5 mM MgCl₂ (14B), and medium (14C), as measured by flow cytometry (Y axis is signal and calculated from the geometric mean (G.M.) from the histogram). Upon addition of PDGF (10 nM), the FAM green signal was quenched.

FIG. 14D is a schematic diagram depicting PDGF aptamer sensor 2 (SEQ ID NOs: 5 and 4) on a cell.

FIG. 15A is a set of histograms depicting representative sensor performance data, examined via flow cytometry, for the quench sensor (sensor 2) immobilized on the MSC surface before and immediately after addition of 10 nM PDGF (G.M.=geometric mean).

FIG. 15B is a graph depicting concentration dependence of fluorescence quenching of sensor 2 on MSCs in response to PDGF (x-axis). The y axis, signal, is the quenching ratio calculated from the geometric mean from flow cytometry analysis.

FIGS. 15C-15D are micrographs depicting sensor performance data before and immediately after addition of 10 nM PDGF in PBS, respectively.

FIG. 15E is a photomicrograph depicting cells following addition of PDGF (2 μM) from the top/left corner (arrow indicates PDGF flow direction) by a pipette tip to sensor modified MSCs.

FIG. 15F is a graph depicting the PDGF gradient from FIG. 15E separated into five regions using image analysis and the fluorescent intensity of 10 representative cells from each region were averaged. The sensor signal in region 1 is defined as 1 and other regions were normalized accordingly.

FIGS. 16A-16C are schematic diagrams of sensors 4 (16A), 5 (16B), and 6 (16C), using Cy3 and Cy5 as FRET donor and receptor, respectively. The distance between cy3 and cy5 in different sensors follows sensor 6>sensor 5>sensor 4. The sensors include SEQ ID NO:5 and SEQ ID NO:4.

FIGS. 17A-17C are fluorescence spectra depicting the performance of sensors 4 (17A), 5 (17B), and 6 (17C) in PBS solution as recorded by fluorometer. In sensor 4, upon adding PDGF (10 nM), both Cy3 (570 nm emission) and Cy5 (670 nm emission) dyes are quenched. In sensors 5 and 6, Cy3 and Cy5 are quenched and enhanced, respectively, upon adding PDGF (10 nM).

FIGS. 18A-18C are bar graphs depicting performance of sensor 5 on MSCs in PBS (18A), PBS with 0.9 mM CaCl₂ and 0.5 mM MgCl₂ (18B), and medium (18C). In all cases, upon addition of PDGF (10 nM), Cy3 fluorescence was quenched and Cy5 fluorescence increased. The Cy3 and Cy5 fluorescence intensity in these data were G.M. from flow cytometry analysis.

FIG. 18D is a schematic diagram of sensor 5 on an MSC cell. The sensor includes SEQ ID NO:5 and SEQ ID NO:4.

FIG. 19A is a schematic diagram of a thrombin aptamer sensor on cells. In this construct, both sensor strands are aptamers. Upon binding to thrombin, the two stands of aptamer bring the attached FRET dyes into close proximity which gives a FRET signal.

FIG. 19B depicts fluorescence spectra of thrombin sensor (10 nM) in PBS with Ca/Mg before and after addition thrombin (5 NIH units/500 μL).

FIG. 20 is a schematic presentation of aptamer-promoted cell-cell interactions. Aptamers serving as adhesion molecules are attached onto cells (Cell 1) using the strategy presented in FIG. 4. The aptamer promotes the interaction of cell with aptamer target coated surfaces or cells that express the aptamer target (Cell 2).

FIGS. 21A-21C are flow cytometry histograms depicting the successful conjugation of an L-selectin binding aptamer (labeled with a FITC dye) on MSCs using biotin-modified aptamers (21A), non-biotin-modified aptamers (21B), and unmodified MSCs without streptavidin (21C).

FIG. 22 is a bar graph depicting static adhesion of L-Aptamer-MSC on an L-selectin-coated substrate with controls including scrambled sequence aptamer modified MSCs on L-selectin, PBS MSC on L-selectin, and L-Aptamer-MSC on P-selectin. The adherent cell numbers in controls were normalized using the number of L-Aptamer-MSC on L-selectin as 100.

FIG. 23 is a line graph depicting results of a flow chamber assay of aptamer-modified MSCs on L-selectin coated cell culture petri dish surfaces together with controls. Specifically, 500,000 cells suspended in MSC medium were infused to the flow chamber and were then allowed to adhere to the surface for 1 min before tuning on the flow. The percent of cells left on the surface from the original cells (Y axis) was plotted as a function of flow rate (X axis). (i) L-Aptamer-MSC on L-selectin coated surfaces (5 μM aptamer was used in the conjugation), (ii) PBS MSC on an L-selectin surface, (iii) L-Aptamer-MSC on a P-selectin surface, (iv) L-Aptamer-MSC on an L-selectin surface in the presence of 5 mM EDTA, (v) scrambled sequence aptamer modified MSC on L-selectin coated surfaces, (vi) L-Aptamer-MSC on L-selectin surface pre-blocked with L-selectin aptamers, (vii) native HL60 cells on L-selectin coated surfaces and (viii) L-Aptamer-MSC (0.5 μM aptamer was used in conjugation) on L-selectin coated surfaces.

FIG. 24A is a line graph depicting tethering of L-selectin aptamer modified MSCs on L-selectin coated surface under flow conditions. The flow rates were applied for 1 minute at each shear rates which started from 0.5 dynes/cm², then 1 dynes/cm², until 20 dynes/cm². The total cell number in the same microscopy view was counted at the end of each shear rate and plotted as Y axis.

FIGS. 24B-24C are representative photomicrographs of tethering for L-Aptamer-MSC on L-selectin coated substrate under continuous flow condition (i.e. cells were not permitted to interact with surface) at 0.75 dyn/cm2. Images were acquired under flow conditions at time 0 (24B) and 5 min (24C).

FIG. 25 is a set of micrographs depicting static adhesion of L-selectin expressing leukocytes isolated from human fresh blood on L-selectin aptamer modified MSC (left), non-aptamer-DNA modified MSC (middle) and unmodified MSCs (right) in both MSC medium (top) and PBS with Ca/Mg (bottom). The modification of aptamer and non-aptamer DNA were directly performed on adhered MSCs. Leukocytes (50,000) were added onto modified and unmodified MSCs, allowed to bind for 1 h at room temperature after which the wells were washed 3 times using PBS with Ca/Mg and microscopy images were then taken. As shown in this figure, leukocytes adhere well on L-selectin aptamer modified MSC in both medium and PBS with Ca/Mg whereas showed little binding on non-aptamer-DNA modified MSC and unmodified MSCs.

FIG. 26 is a line graph depicting results of a flow chamber assay of leukocytes (white blood cells, WBCs) isolated from human fresh blood on L-selectin aptamer modified MSC, non-aptamer-DNA modified MSC and unmodified MSCs. The percent of cells left on the surface from the original cells (Y axis) was plotted as a function of flow rate (X axis).

FIG. 27 is a set of micrographs depicting static adhesion of P-selectin aptamer modified MSCs and unmodified MSCs on P-selectin coated 24 well-plate surfaces. The cells (˜50,000) were allowed to incubate with surfaces for 30 min and washed 3 times by PBS with Ca/Mg. The cells adhered on the surface were then recorded by microscopy which showed that P-selectin aptamer MSC adhere much more on P-selectin coated surface than does unmodified MSCs.

FIG. 28A is a bar graph depicting static adhesion of P-Aptamer-MSC on P-selectin coated substrates compared to controls including scrambled sequence modified MSCs on P-selectin, PBS treated MSCs on P-selectin and P-Aptamer-MSC on L-selectin. The adherent cell numbers in controls were normalized using P-Aptamer-MSC on P-selectin as 100.

FIG. 28B is a line graph depicting results of a flow chamber assay of P-selectin aptamer modified MSCs, non-aptamer-DNA modified MSCs and unmodified MSCs on P-selectin-Fc coated cell culture dish. Specifically, 500,000 cells suspended in MSC medium were infused to the flow chamber and were then allowed to adhere to the surface for 3 min before tuning on the flow. The percent of cells left on the surface from the original cells (Y axis) was plotted as a function of flow rate (X axis). (i) P-Aptamer-MSC on P-selectin surface, (ii) PBS-MSC on P-selectin surface, (iii) scrambled sequence modified MSCs on P-selectin surface, (iv) P-Aptamer-MSC on L-selectin surface, (v) P-Aptamer-MSC on P-selectin surface in the presence of 5 mM EDTA, and (vi) P-Aptamer-MSC on P-selectin surface pre-blocked with P-selectin aptamers. Note that in (b) only the cells that were initially in the field of view were considered. In these experiments, ˜500,000 cells suspended in MSC medium were infused to the flow chamber and were then allowed to adhere to the surface for 3 min before tuning on the flow. The percent of cells left on the surface from the original cells (Y axis) was plotted as a function of flow rate (X axis).

FIG. 29 is a schematic depiction of an Enzyme-linked Aptamer Sorbent Assay (ELASA).

FIGS. 30A-30C are schematic depictions of ultrasensitive detection of cell surface markers using long DNA probes produced by rolling circle amplification (RCA). FIG. 30A depicts RCA is performed on a cell surface in situ. FIG. 30B depicts labeling and detection of cell surface molecules with long DNA molecules produced by RCA and including multiple aptamer units. FIG. 30C depicts long DNA probes on beads and nanoparticles where one particle can contain tens to hundreds of long DNA strands.

FIG. 31A is a schematic diagram depicting production of long DNA molecules on a cell surface in situ and labeling of the long DNA molecules with dyes attached to complementary DNA strands. Labeled, non-complementary DNA strands do not bind.

FIGS. 31B-31C are flow cytometry histograms depicting fluorescence of cells with long DNA molecules produced by RCA and labeled with complementary DNA (31B) or non-complementary DNA (31C).

FIGS. 32A-32F are flow cytometry histograms depicting fluorescence of cells probed with a long DNA produced by RCA and containing multiple L-selectin aptamer units. 32A, unlabeled KG1a. 32B, KG1a labeled with single unit aptamer at 2 μM. 32C, KG1a labeled with single unit aptamer at 35 nM. 32D, KG1a labeled with a long, aptamer-containing DNA probe at 35 nM. 32E, unlabeled non-L-selectin-expressing MSCs. 32F, MSCs labeled with a long DNA probe.

FIGS. 33A-33F are flow cytometry histograms depicting fluorescence of cells probed with a long DNA produced by RCA reactions carried out for varying times. 33A, unlabeled KG1a. 33B, KG1a labeled with probe resulting from 1 minute RCA reaction. 33C, KG1a labeled with probe resulting from 5 minute RCA reaction. 33D, KG1a labeled with probe resulting from 10 minute RCA reaction. 33E, KG1a labeled with probe resulting from 30 minute RCA reaction. 33F, KG1a labeled with probe resulting from 60 minute RCA reaction.

FIGS. 34A-34F are fluorescence micrographs depicting fluorescence of cells probed with a long DNA produced by RCA reactions carried out for varying times. 34A, unlabeled KG1a. 34B, KG1a labeled with probe resulting from 1 minute RCA reaction. 34C, KG1a labeled with probe resulting from 5 minute RCA reaction. 34D, KG1a labeled with probe resulting from 10 minute RCA reaction. 34E, KG1a labeled with probe resulting from 30 minute RCA reaction. 34F, KG1a labeled with probe resulting from 60 minute RCA reaction.

FIG. 35A is a schematic diagram depicting anchoring of an engineered aptamer sensor to a cell surface.

FIGS. 35B-E are flow cytometry histograms depicting successful aptamer sensor conjugation to the cell suface using the Cy3 signal of the FRET sensor as an example.

FIG. 36 is a schematic diagram depicting probing of cellular niches by aptamer engineered cells.

FIG. 37 is a line graph depicting the ratio of fluorescence before and after addition of 10 nM PDGF for engineered and original PDGF sensors in PBS with 0.9 mM CaCl₂ and 0.5 mM MgCl₂.

FIGS. 38A-38B are bar graphs depicting normalized signal of the sensor of FIG. 14D (38A) and the sensor of FIG. 18D (38B). Signal for the quench sensor is defined as the ratio of geometric means of the flow cytometry histogram before and after addition of PDGF. Signal for the FRET sensor is defined as the fluorescence decrease of donor dye (Cy3)×fluorescence increase of acceptor dye (Cy5) based on the geometric means in the flow cytometry histogram. 20 nM PDGF was used.

FIG. 39A is a set of fluorescence micrographs of a single MSC functionalized with a PDGF quench sensor following injection of PDGF (2 μM) 30 μm from the cell via a micro-needle as indicated by the orange arrow. The scale bar represents 10 μm.

FIG. 39B is a representation of the concentration of PGDF on the cell surface as predicted from a three-dimensional computational mass transport model.

FIG. 40A is a diagram of a computational domain used for modeling PDGF transport. The boundary conditions are shown along with their values. The dimensions are also shown. Note that the flow is determined primarily by the direction and magnitude of injection velocity, and pipette body has minimal effect on the flow profile. This allows us to model the pipette as a thin vertical tube (a) and the direction of injection) (30° and magnitude of velocity (100 μm/s) are similar to those used in the experiment.

FIG. 40B is a diagram of a discretized computational domain showing the tetrahedral elements used for meshing.

FIG. 41 is a diagram depicting real-time sensing of PDGF secretion from neighboring MDA-MB-231 cells by sensor-engineered MSCs in the presence of media containing 15% FBS. The left panel shows representative images of microwells containing different number of PDGF-producing MDA-MB-231 (green) in the same well with sensor-MSC (red) at time 0. n is the number of MSCs used in the analysis. Note that MDA-MB-231 is genetically engineered to secrete PDGF that is fused with a GFP tag which is used to track the transduction process. To be distinguishable, the quench sensor attached on MSCs in this set of experiments is labeled with a red-colored dye, Cy5, and Iowa Black RQ as a quencher instead of FAM and Dabcyl used in FIG. 1c. Cy5/Iowa Black RQ and FAM/Dabcyl perform similarly in terms of PDGF induced fluorescence quenching (data not shown). Right panel shows that the fluorescence of MSC engineered with the quench sensor declined during the course of PDGF production. The signal, which is defined as the percentage of MSCs that have fluorescence intensity less than 50% of their initial value at the indicated time, correlates with the number of PDGF-producing MDA-MB-231 cells in the same well as a sensor-MSC.

FIG. 42 is a schematic diagram of a “light up” sensor. In the absence of target molecule PDGF, a short complementary DNA bearing a quencher molecule (Dabcyl) hybridizes with PDGF aptamer attached to fluorescein. The fluorescence is quenched as dye and quencher is at close proximity. In the presence of PDGF, aptamer binds to PDGF and releases complementary DNA strand which moves quencher away from dye and therefore fluorescence increases.

FIGS. 43A-43C are a set of schematic diagrams depicting three types of cell-cell interactions under dynamic flow conditions through engineering the cell surface with aptamers. 43A, flowing cell-1 (P-selectin aptamer-MSC) tethers to adherent cell-2 (P-selectin expressing endothelial cell). 43B, flowing cell-2 (L-selectin expressing leukocyte) tethers to a P-selectin coated substrate using the native leukocyte/P-selectin interaction and then captures flowing cell-1 (L-selectin aptamer-MSC). 43C, cell-1 (L-selectin aptamer-MSC) and cell-2 (L-selectin expressing leukocyte) first complex in the flowing stream and then tether to a P-selectin coated substrate.

FIG. 44A is a schematic illustration depicting the chemical immobilization of aptamers onto the MSC surface using a three-step procedure including biotinylation of cell surface by reacting cell surface NH2 groups with NHS-biotin, subsequent incubation with streptavidin and finally conjugation with biotin-modified aptamers.

FIG. 44B is a histogram depicting successful conjugation of aptamers on MSC surface was confirmed by flow cytometry. A positive fluorescence signal was observed for MSCs modified with aptamer-dye and the intensity of the signal is directly related to the concentration of aptamer used during the conjugation process.

FIG. 45 is a set of micrographs demonstrating aptamer stability/accessibility on MSCs. L-Aptamer-MSCs were cultured on 12 well plates and stained by FAM-antisense at multiple time points to examine the stability and accessibility of the L-selectin aptamer on the cell surface.

FIG. 46A is a bar graph depicting viability of L-Aptamer-MSCs and unmodified PBS-MSCs immediately after modification (0 h) and after 48 hours.

FIG. 46B is a bar graph depicting adherence of L-Aptamer-MSCs and PBS-MSCs measured at 10, 30, and 90 min.

FIG. 46C is a line graph depicting proliferation of L-Aptamer-MSCs and PBS-MSCs over 8 days.

FIG. 46D is a set of micrographs depicting alkaline phosphatase (ALP) and oil red O (ORO) staining 23 days after addition of osteogenic and adipogenic differentiation media, respectively. Negative controls (L-Aptamer-MSCs cultured in expansion media) showed no ORO or ALP staining Positive controls (PBS-MSCs in differentiating media) showed positive ORO and ALP staining Experimental group (L-Aptamer-MSCs in respective differentiating media) showed positive staining for both ORO and ALP. This indicated that aptamer surface modification did not compromise MSC's multilineage differentiation potential.

FIG. 47A is a set of representative micrographs demonstrating the accumulation of P-Aptamer-MSCs on HUVEC when low to high shear stresses were applied: the total number of adhered cells in the same field of view first increases up to 0.75 dyn/cm² and then starts decreasing at higher shear stresses (1-5 dyn/cm²). Cell numbers at 0.25 (i), 0.5 (ii), and 0.75 (iii), 1 (iv), 2 (v) and 5 dyn/cm2 (vi) are 54, 63, 76, 55, 50 and 42, respectively. Note that perfused circular MSCs (white arrow) and adherent spindle-shaped HUVEC (red arrow) can be easily distinguished by their differing shapes.

FIG. 47B is a line graph depicting percentage of adherent MSC as a function of shear stress. In this figure, only MSCs initially present in the field of view were considered. (i) P-Aptamer-MSC, (ii) MSC treated with PBS instead of P-selectin aptamer in the third step of modification, (iii) scrambled sequence modified MSC, and iv) P-Aptamer-MSC on HUVEC pre-blocked with P-selectin aptamers.

FIGS. 48A-48B depict interactions between L-Aptamer-MSCs and neutrophils on a P-selectin substrate under flow conditions at 0.25 dyn/cm². Note that neutrophils and MSCs, which are ˜8 μm and ˜20-25 μm in diameter, respectively (determined by examining pure populations of neutrophils and MSCs by microscopy), can be easily distinguished from each other by size. Representative examples of (48A) an adherent neutrophil (orange arrow) capturing a flowing MSC (blue arrow) and (48B) a neutrophil (orange arrow) complexed with MSC (blue arrow) first in the flowing stream and then tethered onto the P-selectin surface. Note that once captured, MSCs always shift their position to the left of the immobilized neutrophils due to the flow direction (from right to left in this case), which clearly demonstrates that the capture of MSC on P-selectin was mediated by binding of neutrophils to the P-selectin coated substrate versus MSC binding to the P-selectin coated substrate.

FIG. 49 is a representative image of typical L-selectin binding Aptamer modified MSC/neutrophil interactions in the flow stream at a shear stress of 0.25 dyn/cm2. Orange, blue, and red arrows indicate MSC/neutrophil complexes with a MSC:neutrophil ratio of 1:1, 1:2 and 1:3 (or 3+), respectively. Larger cellular aggregates, i.e. comprising two or more MSCs and multiple neutrophils, were also observed and highlighted in boxes.

FIGS. 50A-50C are representative images of (50A) neutrophils and PBS-MSCs, (50B) neutrophils and scrambled sequence modified MSCs and (50C) neutrophils pre-blocked with L-selectin aptamers and L-Aptamer-MSCs, on P-selectin coated surface under flow condition (0.25 dyn/cm² in this figure).

FIG. 51A is a schematic diagram of preparation of long DNA molecules containing multiple aptamer units using rolling circle amplification. In one approach, avidin is first adsorbed onto glass substrate. DNA primer, tethered with biotin is annealed with circular template and subsequently conjugated to the avidin surface. Rolling circle amplification is then conducted in the presence of DNA polymerase (phi29) and deoxyribonucleotide triphosphates at isothermal conditions.

FIG. 51B is a schematic diagram of use of three dimensional, long, multivalent aptamer network to capture circulating cells from a mix of cells under flow conditions.

FIGS. 52A-52E depict parameters that tune RCA product properties and therefore cell capture performance. (52A) The length of RCA product can be adjusted by, for example, the RCA reaction time. (52B) the graft density of RCA products can be tuned by using a dilute molecule (e.g., biotin-modified non-primer). (52C) the conformation of RCA product can be regulated by hybridizing a short complementary DNA strand which is expected to yield a more extended form of RCA product. (52D) Multiple types of aptamers can be incorporated into the RCA product which allows the device to selectively capture and detect one or multiple type of cells. (52E) Captured cells can be released by restriction enzymes which digest DNA from the substrate.

FIG. 53A is a line graphs depicting number of CCRF-CEM cells captured per field of view vs. shear. Shears that were continuously applied from high to low with 1 minute at each shear. Long d.s. sgc.8 aptamer-CCRF CEM: substrate with double-stranded RCA products containing CCRF CEM cell binding aptamers+CCRF CEM. Long s.s. sgc.8 aptamer-CCRF CEM: substrate with single-stranded RCA products containing CCRF CEM cell binding aptamers+CCRF CEM. Unit sgc.8 aptamer-CCRF CEM: substrate with a single unit CCRF CEM cell binding aptamers+CCRF CEM. Long s.s. random-CCRF CEM: substrate with single-stranded RCA products containing scrambled DNA sequences+CCRF CEM. Long d.s. sgc.8 aptamer-Romas: substrate with double-stranded RCA products containing CCRF CEM cell binding aptamers+Romas (control cell). Unit random DNA-CCRF CEM: substrate with a single unit scrambled DNA+CCRF CEM.

FIG. 53B is a line graph depicting number of cell captured per field of view vs. capture time. Shear is fixed at 1 dynes/cm².

FIG. 54 is a line graph depicting attachment of cells by a long RCA product containing multiple DNA aptamers holds under shear more strongly than monovalent aptamer. Percentages of cancer cells that remained per field of view (Y axis) after rinsed at increasing shear forces (X axis).

### DETAILED DESCRIPTION

### Introduction

The present disclosure describes, among other things, methods of engineering cells with aptamers and the uses thereof. The immobilization of aptamers on cell membranes includes, but is not limited to, a covalent method where a stepwise NHS-biotin treatment, streptavidin, and biotin-aptamer modification process is applied. In the case of aptamer sensor-engineered cells, sensors on cell membranes enable the real-time detection of molecules present in the cell medium, and the study of cells' local nanoenvironment and niche (e.g., in vivo), cell-cell communications and signaling, and in vivo cell trafficking, homing, and differentiation. The disclosure also describes methods of engineering existing aptamer sensors to be suited for cell surface immobilization, for proper function at physiological conditions, and for improved detection signals. In one embodiment, the present disclosure describes aptamer sensors on MSCs that can detect PDGF and thrombin in real time in situ. Fluorescent dyes and quenchers can be used as signal transducers in a fluorescence quenching or FRET assay. The disclosure also describes methods of multiplex sensing assays using immobilized multiple sensors on the same or different cells that detect analytes simultaneously. The disclosure also provides methods of using sensor-modified beads for facile detection of cytokines.

Further included in the present disclosure are methods of engineering cells with aptamers that can promote a desirable cell-cell interaction, and cell adhesion such as cell rolling and/or firm adhesion under both static and flow conditions. The present disclosure includes, but is not limited to, aptamer-engineered cells that can bind to L- or P-selectin expressing cells. Specifically, L-selectin aptamer engineered MSCs bind strongly to L-selectin-coated surfaces or L-selectin expressing leukocytes. In a similar manner, P-selectin aptamer engineered MSCs bind to P-selectin coated surfaces.

The present disclosure also describes methods of using enzyme-linked aptamer sorbent assays (ELASA) for facile, multiplex, high throughput and ultrasensitive detection of markers present in the biological solutions. In the present disclosure, nucleic acid aptamers are used as target recognition molecules. The signal can be amplified by two enzyme reactions, e.g., RCA that converts a single binding event to a long DNA molecule that contains hundreds of DNA enzyme units. In a second signal amplification step, a DNA enzyme capable of multiple turnovers converts chromogenic or fluorogenic dyes to color signal or fluorescence signal. The present disclosure includes, but is not limited to, an ELASA for PDGF detection.

The present disclosure also describes methods of using long DNA probes that are labeled with dyes for the ultrasensitive detection of cell surface markers. These long DNA molecules, e.g., produced by RCA, can be synthesized and stained on cell surfaces in situ or in solution first and then labeled on cells. Essentially, the detection of a single cell surface marker is feasible using this method.

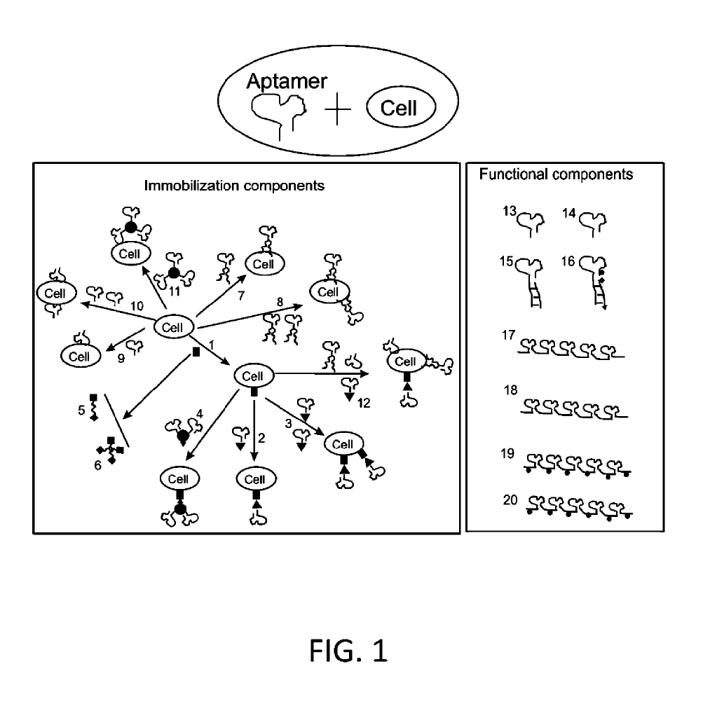

Referring to FIG. 1, various functional components of aptamer engineered cells as disclosed herein are shown. 1. The membrane of a cell is functionalized (e.g., covalently) to introduce a specific surface functional group. 2. Aptamers that include a second functional group can be attached to a cell having a surface functional group. 3. Different aptamers can be co-attached to cells via functional groups. 4. One or more types of aptamers can be attached to a support bead and then attached to cells via functional groups on the bead. 5. Cross-linkers and/or spacers can be used for attachment of functional groups. 6. Functional groups used for modifying cells can be branched or star-shaped in some embodiments. 7. Lipid-modified aptamers can be attached to unmodified cells, e.g., by self-assembly. 8. Different types of lipid-modified aptamers can be co-attached to unmodified cells. 9. Aptamers can be attached to unmodified cells via non-covalent, biological interactions between aptamers and markers on the unmodified cells.

10. Different types of aptamers can be co-attached to cells via biological interactions. 11. Aptamers (e.g., the same or different types of aptamers) that are attached on a bead support can be attached to cells via non-covalent, biological interactions between an aptamer and markers on unmodified cells. 12. Aptamers (e.g., the same or different types of aptamers) can be attached to cells via any combination of covalent and noncovalent conjugations. 13. Aptamer constructs in the present invention can be single stranded, 14. A different aptamer that binds to a different target. 15. Aptamer constructs in the present disclosure can be also double stranded. 16. Aptamer constructs can be modified with functional moieties including, e.g., dyes and biotin. 17. Long nucleic acid strands with multiple aptamers and/or DNA enzymes can be produced. 18. The long nucleic acid strands can include multiple different types of aptamers and/or DNA enzymes. 19. The long nucleic acids that contain multiple aptamers and/or DNA enzymes can be labeled with one or more dyes or other moieties. 20. Long nucleic acids that contain multiple different aptamers and/or DNA enzymes can be labeled with one or more dyes or other moieties.

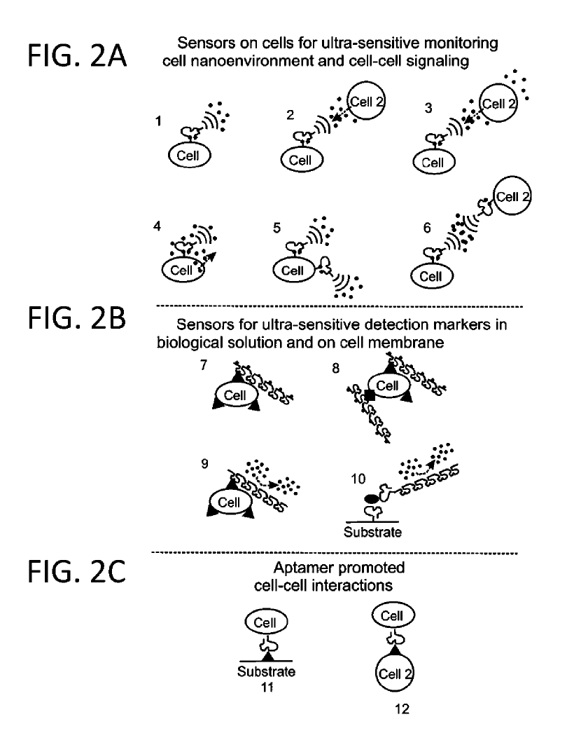

FIGS. 2A-2C illustrate various methods of using the compositions disclosed herein. FIG. 2A depicts uses of cells with aptamer sensors for monitoring the cell nanoenvironment and cell-cell signaling. 1. A cell with an attached aptamer is used to monitor the presence of target molecules in the cell surface nanoenvironment. 2. A cell with an attached aptamer is used to monitor the release of target molecules from a different cell. 3. A cell with an attached aptamer is used to monitor the release of target molecules from a different cell triggered by a second molecule. 4. A cell with an attached aptamer is used to monitor the release of target molecules from the same cell. 5. A cell with multiple aptamer sensors attached is used for monitoring multiple targets at the same time. 6. Multiple aptamer sensors are attached on different cells for monitoring of multiple targets at the same time. FIG. 2B depicts uses of long nucleic acids that include multiple aptamer units for detection of markers in biological solutions and on cell membranes. 7. Long DNA probes that contain aptamers and are labeled with multiple dyes (e.g., the same or different dyes) are used to stain and detect cell surface markers. 8. Long DNA probes that contain different types of aptamers are used for detection of different cell surface markers at the same time. 9. Long DNA probes that contain DNA enzymes that convert chromogenic dyes to color signal and/or aptamers are used for detection of markers. 10. An enzyme linked aptamer sorbent is used for ultrasensitive detection of molecules present in biological solutions. In FIG. 2C, use of aptamers to promote cell binding are depicted. 11. Aptamers on cells promote binding between the cell and a substrate. 12. Aptamers on cells promote binding between cells.

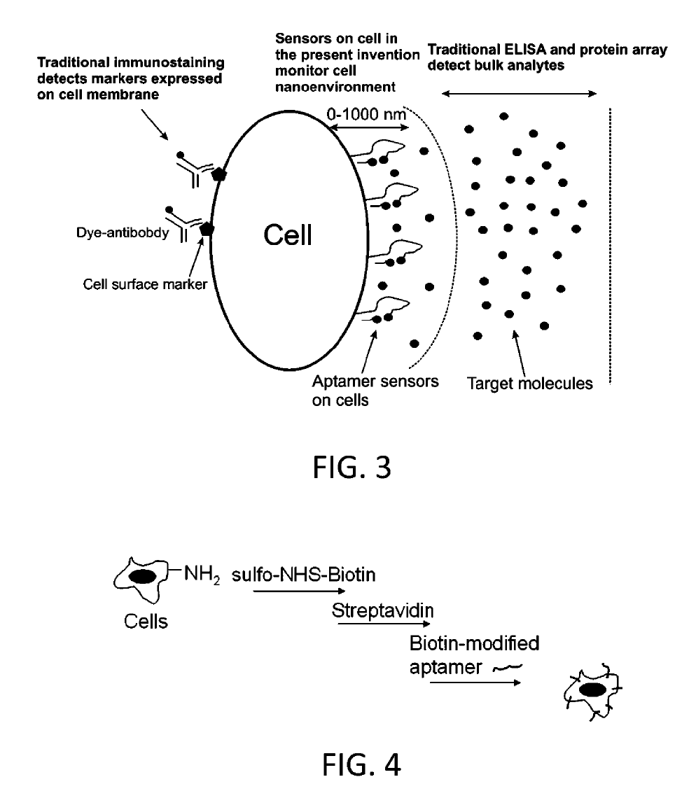

Compositions that include aptamers immobilized on cells can be used to monitor the nanoenvironment of the cell (e.g., the environment 0-1000 nm from the cell surface). Referring to FIG. 3, it depicts immunostaining to detect markers expressed on the cell membrane and traditional ELISA and protein array methods, which detect bulk analytes in solution. Aptamer sensors on cells enable the monitoring of the cell nanoenvironment.

### Aptamers

Nucleic acid aptamers are typically single-stranded DNA or RNA molecules that can specifically bind to a non-nucleic acid target including protein, small molecule, metal ion, and cell, etc. Aptamers that bind to a specific target can be isolated, e.g., using in vitro SELEX method, and are typically 15-100 nucleotides long. Klussmann, S. The Aptamer Handbook Functional Oligonucleotides and Their Applications, 2006, WILEY-VCH, Weinheim, provides a comprehensive review of aptamers and their selection, production, and uses. Additional information regarding aptamers can be found, e.g., in Ellington et al., 1990, Nature, 346:818; Joyce, 1989, Gene, 82:83-87; and Tuerk et al., 1990, Science, 249:505. Aptamers, as specific binders, have some appealing features compared to antibodies including 1) high binding affinity and high specificity, 2) capability of generation using a bench top procedure, and therefore the properties of aptamer to be selected can be pre-defined, 3) synthesis by scalable and reproducible chemical processes, 4) long shelf-life time, 5) little cytotoxicity and low immunoresponse, 6) relatively small size, 7) and high engineerability such that they can be modified with a number of functionalities (e.g., biotin, fluorophore, etc.) during or after synthesis.

Aptamers have been used as therapeutic drugs where they bind to specific biological markers and then block their functions. The first aptamer drug pegaptanib, which binds to VEGF, was granted approval in 2007 for the treatment of age-related macular degeneration (AMD). Aptamers can also be engineered as biosensors in a number of biosensing platforms including fluorescent, electrochemical, and colorimetric detections (Navani et al., 2006, Curr. Opin. Chem. Biol., 10:272-281). For instance, in a (fluorescence resonance energy transfer) FRET assay, two dyes labeled on each ends of an aptamer molecule can communicate and give a signal upon binding to the target, wherein the conformation of the aptamer changes, thus changing the distance of the dyes (Fang et al., 2003, ChemBioChem, 4:829-834; Vicens et al., 2005, ChemBioChem, 6:900-907). Aptamers can also be immobilized onto solid surfaces (e.g., glass, gold substrate, polymer beads, silicon substrate) using standard bioconjugation chemistry. Immobilized aptamers can be used, e.g., for protein purification, biosensing assays, cell isolation, and facilitating cell binding to solid surface (see Klussmann, supra).

Aptamers can be composed of nucleic acids (e.g., ribonucleic acids and/or deoxyribonucleic acids), and can also be modified. As discussed below, aptamers can be modified with anchoring moieties and can also be modified (e.g., during the synthesis process) to include a variety of functional groups including dyes, modified nucleotides, inverted nucleotides (e.g., T) (see US 2005/0096290), polyethylene glycol (PEG), etc. In some embodiments, the aptamers are modified for a particular purpose such as enhancing nuclease resistance.

Aptamers can be selected for a specific target. Optionally, previously identified aptamers can be used in the compositions and methods disclosed herein. Aptamers have been identified that bind to several proteins, including cytokines/growth factors (e.g., vascular endothelial growth factor (VEGF), human interferon gamma, angiopoitein-2, basic fibroblastic growth factor, platelet-derived growth factor (PDGF)), nucleic acid binding proteins (e.g., HIV-1 Tat, HIV-1 Rev, HIV reverse transcriptase, transcription factor E2f, nuclear factor kappa B), serine proteases (e.g., hepatitis C virus-NS3, human neutrophil elastase, thrombin, factor VIIa, factor IXa), antibodies/immunoglobulins (e.g., immunoglobulin E, cytotoxic T cell antigen 4), cell surface receptors/cell adhesion molecules (e.g., P-selectin, L-selectin, prostate-specific membrane antigen), complement proteins (e.g., human complement 5), extracellular membrane proteins (e.g., tenascin-C), lipoproteins (e.g., human non-pancreatic secretory phospholipase A2), and peptides (e.g., ghrelin, neuropeptide calcitonin gene-related peptide 1, gonadotropin-releasing hormone, neuropeptide nociception/orphanin FQ).

In one embodiment of the present invention, aptamers attached to cell membrane are sensors. The aptamer sensors produce signal readout upon specific binding to the target molecule. The signal readouts include, but are not limited to, fluorescence which can be monitored, for example, by standard flow cytometry and microscopy. Other aptamer-based detection platforms including MRI, colorimetric, electrochemical systems, etc. can also be used. In the present invention, fluorescence signal is produced when the distance of two dye molecules (attached to sensor molecules) change, triggered by the aptamer conformational change when binding to its target. The present disclosure includes a fluorescent quenching methods where fluorescence dyes (e.g., FAM, Alexa 488, Cy5) are quenched by a quencher molecule (e.g., dabcyl, Iowa Black RQ) when the target molecule is present or when the target molecule is absent. Other dyes and quenchers are well known in the art and can also be used. The present disclosure also includes a FRET methods where FRET donor molecules (e.g., Cy3, FAM) and FRET acceptor molecules (e.g., Cy5, Cy5.5, TAMRA) communicate with each other and produce signal when the target molecule is present or when the target molecule is absent. Other FRET dye pairs that are well known in the art can also be used. The FRET signal can include the decrease of donor dye fluorescence and/or the increase or decrease of acceptor dye fluorescence. The signal can be interpreted by the fluorescence change of each individual dye, the ratio of such changes, and/or FRET energy transfer efficiency, among other fluorescence methods that are well-known in the art. FRET energy transfer efficiency between the two dyes can be tuned by defining the positions of two dyes on aptamer sensors to therefore improve sensor performance (see Nagatoishi et al., 2006, ChemBioChem, 7:1730-37).

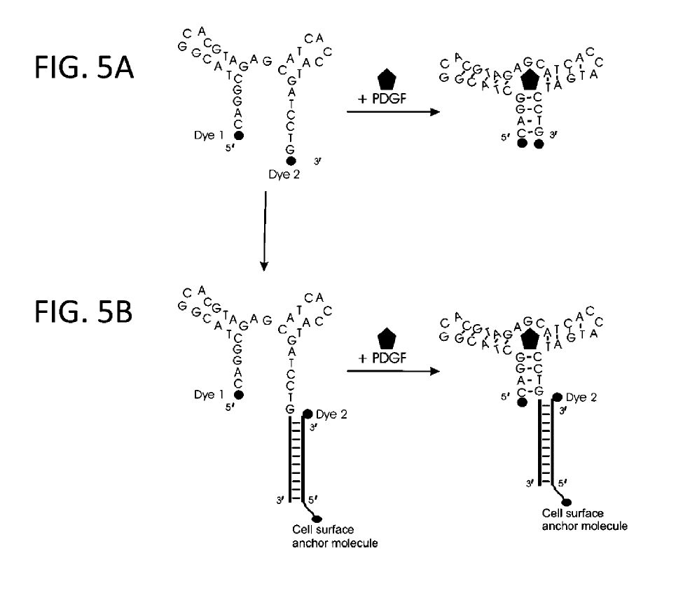

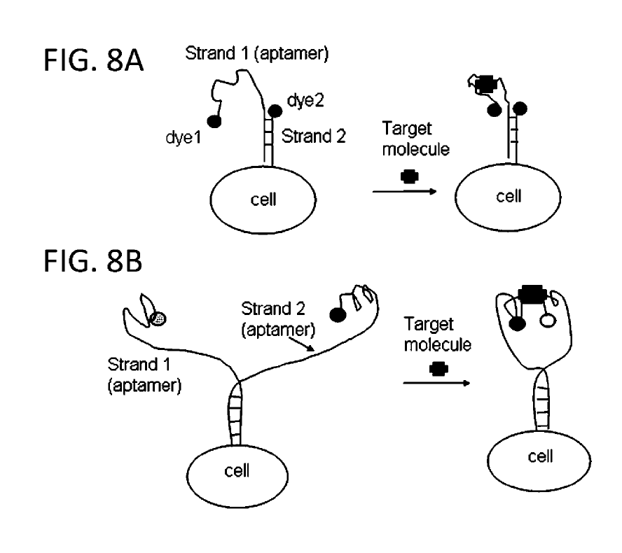

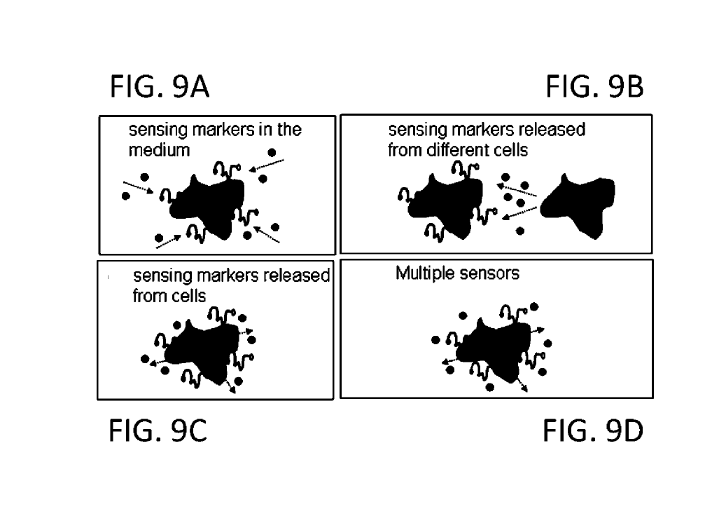

In some embodiments, aptamers can be modified to be cell surface adaptable sensors. In one example, a single stranded aptamer is extended at one end with a short oligonucleotide that can hybridize with a complementary oligonucleotide strand (see, e.g., FIGS. 5A-5B). Two dyes, at desirable positions, and anchor moieties (e.g., biotin or a lipid) can be incorporated (e.g., during synthesis) on these two separated strands, which can be then annealed together before attaching onto cells. In the present disclosure, the two stands in the sensors can each be aptamers, in which case they both can have extended oligonucleotides that hybridize to each other (see FIG. 8B). Anchor molecules can be placed at the end of the duplex oligonucleotide domain, which can allow the sensor to be attached onto cell membrane. The dyes can be modified at the end of each aptamer molecule which both bind to target molecule, which changes the distance between two dyes and produce a fluorescent readout. The present disclosure includes specific PDGF and thrombin aptamer sensors.

PDGF, a dimeric molecule consisting of disulfide-bonded, structurally similar A- and B-polypeptide chains, is a major mitogen for connective tissue cells and certain other cell types. PDGF has great implications of many cell and tissue functions (Heldin et al., 1999, Physiol. Rev., 79:1283-1316). For instance, PDGF signaling leads to stimulation of cell growth, and changes in cell shape and motility. For example, PDGF signaling is important for differentiation and growth of MSCs (Ng et al., 2008, Blood, 15:217-218). PDGF plays important roles in regulating ECs, cancer cells and MSC communications in angiogenesis, tumor growth, etc. (Beckermann et al., 2008, Br. J. Cancer, 99:622-631).

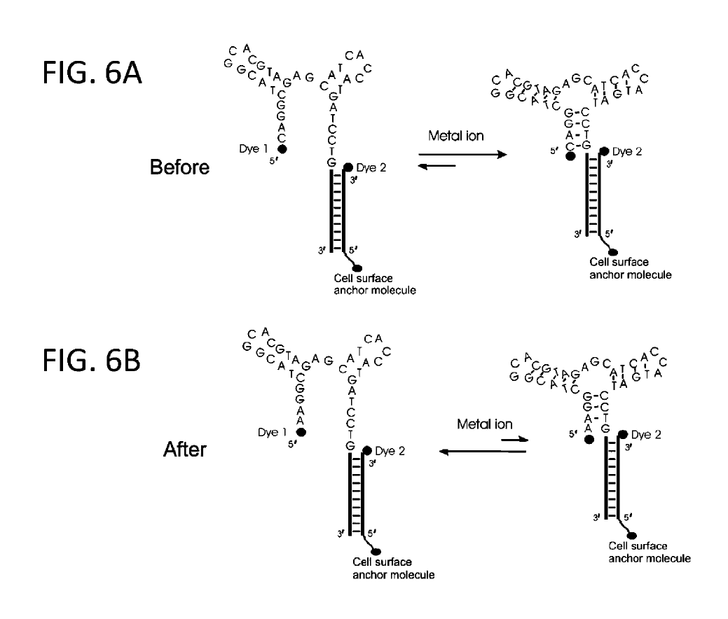

The present disclosure also includes methods of engineering aptamers and aptamer sensors to be more functional under physiological conditions. Aptamer sensors often do not function well in the presence of divalent metal ions such as Ca²⁺ and Mg²⁺, which limits their use in biological systems. Upon binding to the target molecules, aptamers often fold into tertiary structures that include the aptamer binding sequence/target molecule complex and in many cases a duplex nucleic acid domain that stabilizes the formed aptamer/target complex. The stability of such a duplex defines the equilibrium of aptamer folding and unfolding. If the duplex is too stable, in the presence of divalent metal ions for example, the aptamers tend to fold even in the absence of target molecules, which therefore gives a high background signal and low signal/noise ratio. The present invention includes methods of altering the aptamer folding and unfolding equilibrium by altering (e.g., reducing) the length of nucleic acid duplex domain. In one example, by changing a C-G base pair in an existing PDGF aptamer sensor to A-G, the PDGF sensor was able to function better in the presence of divalent metal ions and in growth medium (see, e.g., FIGS. 6A-6B).

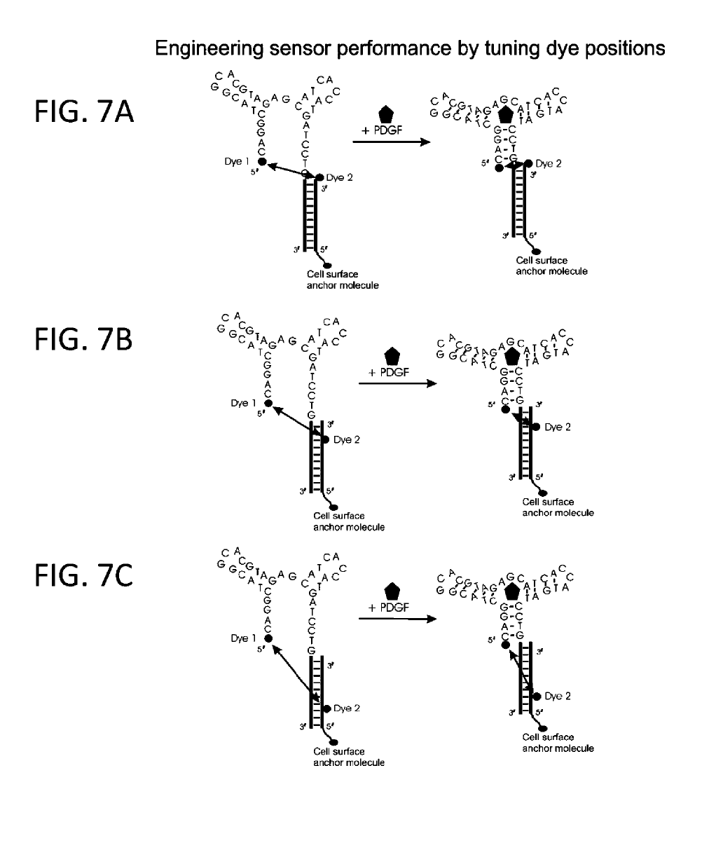

The present disclosure also includes methods of optimizing a FRET signal of an aptamer (e.g., a PDGF aptamer sensor) by altering the positions of the dyes on the sensor strands. In some cases, when the FRET donor dye and acceptor dye were too close to each other, fluorescence quenching was observed for both dyes when the target was added. By placing dyes at farther positions, fluorescence decrease and increase were observed for FRET donor dye and acceptor dye, respectively (see, e.g., FIGS. 7A-7C). This fluorescence increase or “light-up” sensor is useful for certain applications including monitoring cell fate in vivo.

Engineering strategies to allow aptamers to be immobilized onto cell membranes and to be functional well with desirable fluorescence readouts under physiological conditions can be widely applicable to other aptamers. The methods describe herein can be used for attaching a variety of aptamer sensors with desirable properties on cell membranes for given purposes.

Note that people skilled in the art can easily adapt the methods described herein to other cell (or bead) types, other aptamers, and other target molecules for a given application related to (multiplex, high throughput) detection of molecules present in the medium and/or in vivo niche, study cell surface nanoenvironment, and cell-cell communications. The present methods can also be integrated with other biodetection methods including but not limited to MRI, SERS, electrochemical and colorimetric methods. Other commonly biosensing components including gold nanoparticles, quantum dots and carbon nanotubes can also be integrated with the present method to build multi-functional platforms.

### Cells

Essentially any cell can be used in the methods and compositions described herein. For animal use it is preferred that the cell is of animal origin, while for human use it is preferred that the cell is a human cell; in each case an autologous cell source is preferred, although an allogeneic or xenogeneic cell source can be utilized. The cell can be a primary cell, e.g., a primary hepatocyte, a primary neuronal cell, a primary myoblast, a primary mesenchymal stem cell, primary progenitor cell, or it can be a cell of an established cell line. It is not necessary that the cell be capable of undergoing cell division; a terminally differentiated cell can be used in the methods described herein. In this context, the cell can be of any cell type including, but not limited to, epithelial, endothelial, neuronal, adipose, cardiac, skeletal muscle, fibroblast, immune cells (e.g., dendritic cells), hepatic, splenic, lung, circulating blood cells, platelets, reproductive cells, gastrointestinal, renal, bone marrow, and pancreatic cells. The cell can be a cell line, a stem cell (e.g., a mesenchymal stem cell), or a primary cell isolated from any tissue including, but not limited to brain, liver, lung, gut, stomach, fat, muscle, testes, uterus, ovary, skin, spleen, endocrine organ and bone, etc.

Where the cell is maintained under in vitro conditions, conventional tissue culture conditions and methods can be used, and are known to those of skill in the art. Isolation and culture methods for various cells are well within the knowledge of one skilled in the art.

In addition, both heterogeneous and homogeneous cell populations are contemplated for use with the methods and compositions described herein. In addition, aggregates of cells, cells attached to or encapsulated within particles, cells within injectable delivery vehicles such as hydrogels, and cells attached to transplantable substrates including scaffolds are contemplated for use with the methods and compositions described herein.

MSCs are connective tissue progenitor cells that have immediate clinical utility for cell-based therapy (MSCs are currently being examined in multiple phase I-III clinical trials to treat a wide variety of diseases) (Ohnishi et al., 2007, Int. J. Hematol., 86:17-21). MSCs represent a potent source of postnatal cells that can be conveniently isolated autologously or used from an allogeneic source without compromising the host immune response. Given their potential for multi-lineage differentiation (Pittenger et al., 1999, Science, 284:143-147) followed by trophic activity and their ability to reduce inflammation through secretion of paracrine factors, MSCs are currently being investigated in clinical trials to restore tissue function for a number of diseases including cardiovascular disease, brain and spinal cord injury, cartilage and bone injury, Crohn's disease and graft-versus-host disease (Sykova et al., 2006, Cell Mol. Neurobiol., 26:1113-29; Filho Cerruti et al., 2007, Artif. Organs, 31:268-273; Gupta et al., 2007, Spine, 32:720-726; Garcia-Olmo et al., 2005, Dis. Colon Rectum, 48:1416-23; Maitra et al., 2004, Bone Marrow Transplant. 33:597-604). However, a significant barrier to the effective implementation of cell therapies is the inability to target these cells with high efficiency to tissues of interest due to the lack of key adhesion molecules on the MSCs (Kawada et al., 2004, Blood, 104:3581-87). The present compositions and methods can be used to provide MSCs that adhere to selectins or other cell surface antigens.

Aptamers can be immobilized onto the cell membrane. The immobilization strategies include, but are not limited to, covalent conjugation methods. Non-covalent conjugation methods such as self-assembly of lipid-conjugated DNA onto cell membrane can be easily performed as well. In some embodiments, the covalent conjugation methods include conjugating a functional group to the cell using a reactive group such as NHS. In exemplary methods, the covalent conjugation methods include a 3 step process including 1) treating cells with a functional moiety (NHS-biotin, 2) streptavidin conjugation and 3) addition of biotin-modified aptamers (see FIG. 4). Parameters such as reagent concentrations, and reaction time can be varied to tune the site density of attached aptamers. Linker molecules such as PEG can be introduced between NHS and biotin, and between biotin and aptamer in order to, for example, enhance the accessibility of attached aptamers. Other covalent conjugation methods for attaching aptamers on cells can also include, for example, NHS-modified aptamers and NH2 groups on cell membrane, HS-modified aptamers with HS groups on cell membranes and phosphine modified DNA and azide-sugar on cells (Chandra et al., 2006, Angew. Chem. Int. Ed., 45:896-901). Methods of functionalizing the cell surface are also described in Zhao et al., 2010, Materials Today, 13:14-21.

### Solid Supports

In some embodiments, the present disclosure includes solid supports (e.g., beads, plates, micro-/nano-particles, etc.) that have attached to them aptamer sensors. The present disclosure also includes methods of using aptamer sensor (e.g., optimized PDGF aptamer sensor)-attached solid supports for detection of targets in cell culture medium. The use of beads or particles can allow for assays using, e.g., flow cytometry and/or microscopy. In the present disclosure, aptamer sensor modified solid supports can be used for multiplex, high-throughput monitoring markers present in cell culture medium, cell-cell communications, drug screening, etc. The aptamer sensor-modified beads can be used separately or integrated to conventional immuno-bead flow cytometry-based bio-analysis.

### Detection Methods

Aptamer sensors on cells as disclosed herein can be used for real-time, in situ study of the cellular nanoenvironment and cell niche. The presence of target molecules in the nanoenvironment (0-1000 nm) of the cell surface activates the sensors on the cell membrane. The sensor-modified cells can be used to detect any target molecule. In one embodiment, the present disclosure includes specific PDGF sensor-modified cells that detect PDGF. The response is specific and target concentration dependent. The detection limit of the current system is about 400 μM of PDGF. The signal is observed very rapidly, i.e., within a few seconds. The present invention also includes thrombin sensor-modified cells that specifically detect thrombin in the medium.

The present invention includes methods of using aptamer sensor-modified cells for study and high throughput analysis of cell-cell interactions. Specifically, sensors on one cell enable the real-time in situ detection of molecules released from other cells. The sensor-modified cells can be used for the detection of any target molecule released from a cell. In one embodiment, the present disclosure includes specific PDGF aptamer sensor-modified MSCs that can detect PDGF released from cells (e.g., ECs, platelets) upon activation by, for example, thrombin.

The present disclosure also includes methods of using aptamer sensor-modified cells for the detection of molecules released from the same aptamer sensor-modified cells upon activation. Any molecule that is released from a cell (e.g., upon a stimulus) can be detected by aptamer sensors on the cell surface. In this aspect, the sensor signal can indicate important cell functions such as activation of the cells. In one embodiment, the present disclosure includes specific PDGF aptamer sensor-modified ECs (or platelets) which can detect PDGF released from the same cells upon activation by, for example, thrombin.

The present disclosure also includes methods of attaching multiple aptamer sensors on the same cells or on different cells. Multiple sensors enable monitoring of multiple target molecules present in the system (and therefore multiple biological functions) at the same time. In the present disclosure, when multiple sensors are attached on same and/or different cells, these cells can not only monitor different molecules that present in the cell nanoenvironment but also indicate the timing of their presence by response at different time points.

In the present disclosure, the capability of sensor-carrying cells permits a new dimension for high throughput drug screening to examine, for example, the impact of drugs to promote cell communication leading to specific biological response. While most high throughput drug screening studies focus on a single cell type, the technology presented in the present invention enables rapid screening of cell-cell communication, which can be used to examine the impact of drugs indirectly. For example, a drug induces cell type A to release factor X, which interacts with cell type B, leading to the release of factor Y. In this example, the sensing systems can be used to examine the release of factors X and Y in real time.

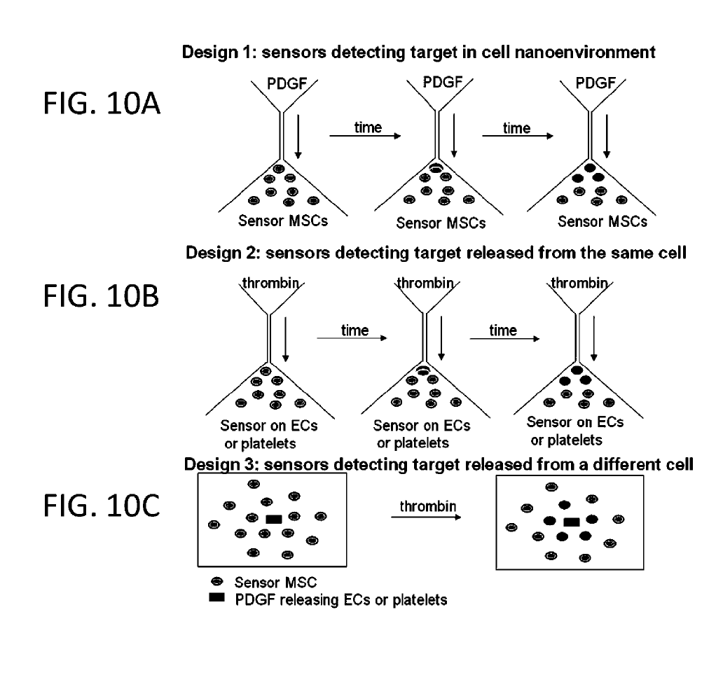

In the present disclosure, the monitoring of cell nanoenvironment and cell-cell communication using cell surface attached aptamer sensors can be facilitated by microfluidic devices. The present invention includes methods of defining target molecule concentration profiles using nanochannels in a microfluidic device. See FIGS. 10A-10B. This enables quantitative study of how sensors on cell membrane respond to the target molecules in the nano-scale frame on the cell surface. In the present invention, target molecules (e.g., PDGF and/or thrombin) are infused from one side of a nanochannel, and diffuse to (e.g., in a fully defined manner), the other side of channel where sensor-modified cells are present. Detection of the concentration of the target molecules in both spatial and temporal dimensions can be monitored. Various nanochannel and microfluidic device configurations are known and can be used to study cell nanoenvironments and cell-cell communication using the cell surface attached aptamer sensors described herein.

In the present disclosure, aptamer sensors on cell membranes enable the monitoring of cell fate (e.g., cell trafficking, homing and/or differentiation) in vivo. In particular, aptamer sensors on cells can enable the detection of target molecules in cell niches in vivo, the study of how cells function in those niches, and how cells communicate to each other in niches. In some embodiments, specific PDGF aptamer sensor modified MSCs can be used to monitor the presence of PDGF in a particular in vivo cell niche and how MSCs communicate with other cells, including ECs and cancer cells, in the niche via PDGF signaling.

### Detection of Markers in Solutions

The present disclosure also includes methods of detecting markers in solutions (e.g., biological solutions) using an Enzyme-linked Aptamer Sorbent Assay (ELASA).

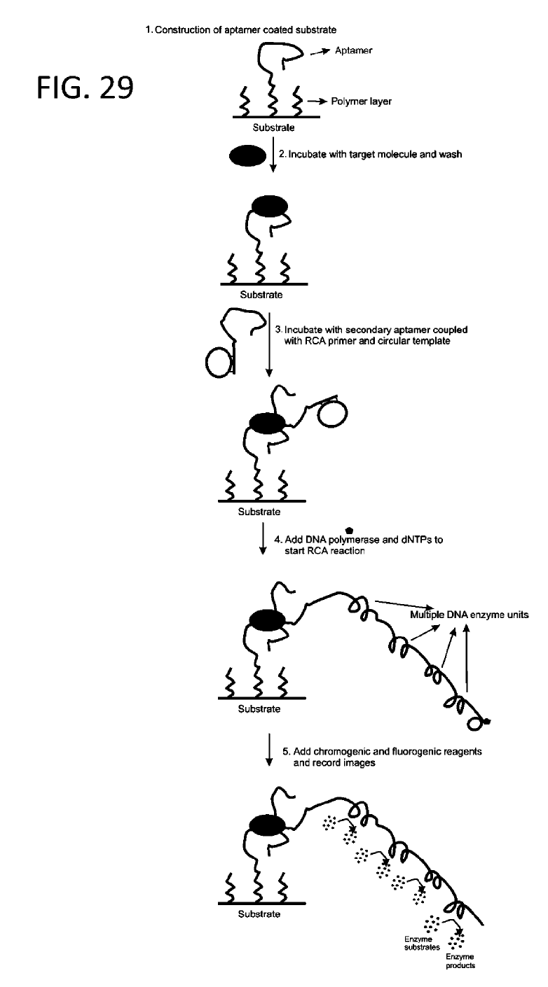

Referring to FIG. 29, aptamer-coated substrates can be provided or constructed for use in the assays at step 1. The substrates can be any standard and widely used substrates such as glass, silicon, gold, etc., or any types of bead or nanoparticles. The coating chemistry includes, but is not limited to Au-thiol chemistry, silane chemistry, streptavidin/biotin interaction, etc. Other polymer molecules such as PEG can be co-immobilized onto the surface for purposes such as preventing nonspecific binding. In step 2, target molecules are added and allowed to bind to aptamer, after which washing steps are applied. In step 3, a secondary aptamer that is coupled with an RCA primer and circular template is added. The aptamer domain will bind to the target, and the RCA primer/circular template will be used for the subsequent RCA reaction. After the aptamer binds to the target and washing step, in step 4, DNA polymerase (e.g., phi29 DNA polymerase) will be added to initialize the RCA reaction in the presence of dNTPs (dATP, dTTP, dCTP and dGTP). The reaction is allowed to proceed, producing potentially hundreds of copies of DNA units (e.g., including DNA enzymes), which in a following step (step 5) convert chromogenic (e.g., 2,2′-Azino-bis(3-ethylbenzthiazoline-6-sulfonic Acid), hemin, luminol) or fluorogenic dyes to color signal or fluorescence signal that can be read by standard colorimetric plate reader, fluorescence reader, or microscopy (Zhao et al., 2008, Angew. Chem. Int. Ed. Engl., 47:6330-37). The present disclosure includes a specific ELASA for the detection of PDGF present in the biological solutions.

RCA is a biological process wherein DNA polymerase elongates DNA or RNA molecules starting from a primer molecule using a circular DNA template (Fire et al., 1995, Proc. Natl. Acad. Sci. USA, 92:4641-45; Rubin et al., 1995, Nucl. Acids Res., 23:3547-53). RCA generates long nucleic molecules that are normally several hundreds of nanometers to microns in length. As the replication is based on the same circular template, the long DNA product contains multiple repeating units.

RCA can be used as an amplification tool for the detection of proteins and nucleic acids where typically antibody coupled primer is used for target binding and RCA initiation (Nilsson et al., 2006, Trends Biotechnol., 24:83-88). Fluorescence dyes attached to the complementary DNA strands can be used to stain the long DNA products. RCA can also be used to produce multiple aptamer units that templates for nanoassembly of proteins based on aptamer/protein binding. RCA can also be used to produce multiple DNA enzyme units that can are capable of converting chromogenic substrates to color products (Zhao et al., 2008, Angew. Chem. Int. Ed. Engl., 47:6330-37). Examples of catalytic nucleic acids can be found in Li and Lu, eds., Functional Nucleic Acids for Analytical Applications, Springer, 2009.

The present disclosure provides the first demonstration of an integrated sandwich assay where aptamer is used as recognition moiety, DNA primer/circular template coupled to aptamer is used to initiate RCA reaction, RCA is used for the first amplification step to produce long DNA with multiple DNA enzyme units which provide a second signal amplification.

In the present methods, nucleic acid aptamers are used as target recognition molecules. As aptamers are more stable than antibodies and have longer shelf-life, this assay will be particularly useful for developing countries where refrigerators are not widely available. The signal is amplified by two enzyme reactions, RCA that converts a single binding event to a long DNA molecule that contains hundreds of DNA enzyme units. In a second amplification step, DNA enzyme that has multiple turnovers converts chromogenic or fluorogenic dyes to color signal or fluorescence signal. Furthermore, the overall assay time of the present method is about 1 hour, which is much shorter than a typical ELISA (˜4 hours or longer).

The present methods can be easily formulated to high throughput assays for multiplex analysis. People skilled in the art can use the present assay for the detection of virtually any target molecule.

### Detection of Cell Surface Markers

The present disclosure includes methods for ultrasensitive detection of cell surface markers using long DNA probes produced by RCA. Specifically, these long DNA molecules contain hundreds of aptamer units and hundreds of dyes, which can lead to massive signal amplification when one strand binds to the marker on cell surface. As long DNA probes appear as super bright dots on cell surface, this method is particularly useful for single surface marker detection or mapping the surface marker distribution on cells.

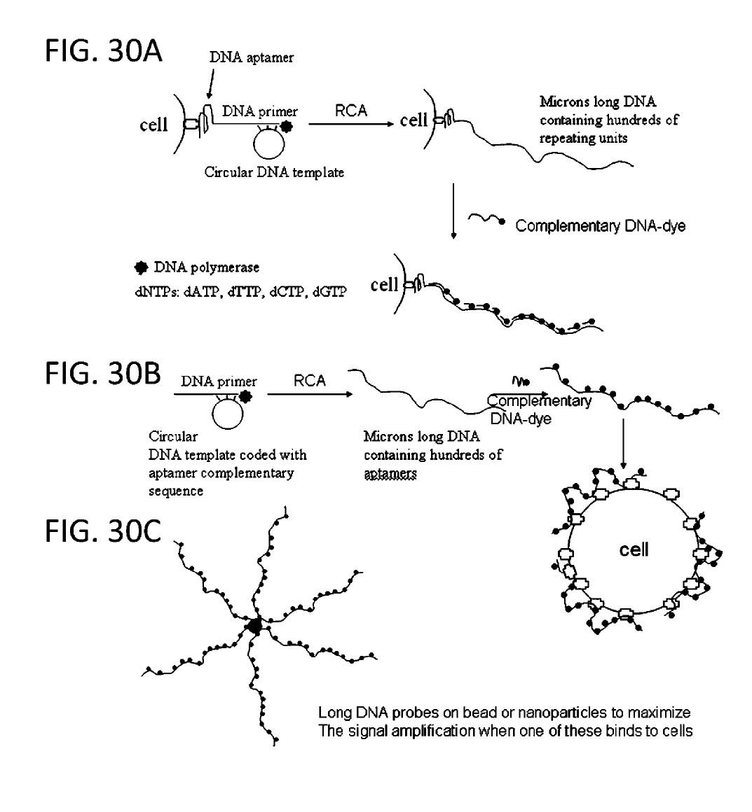

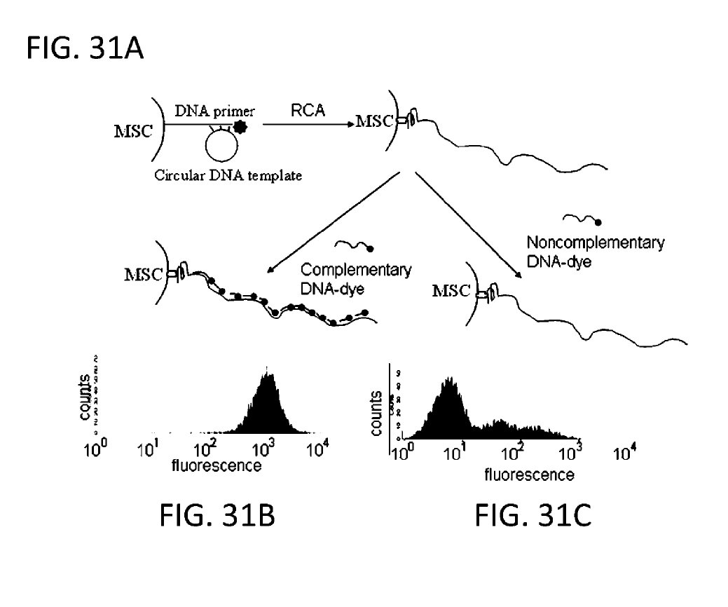

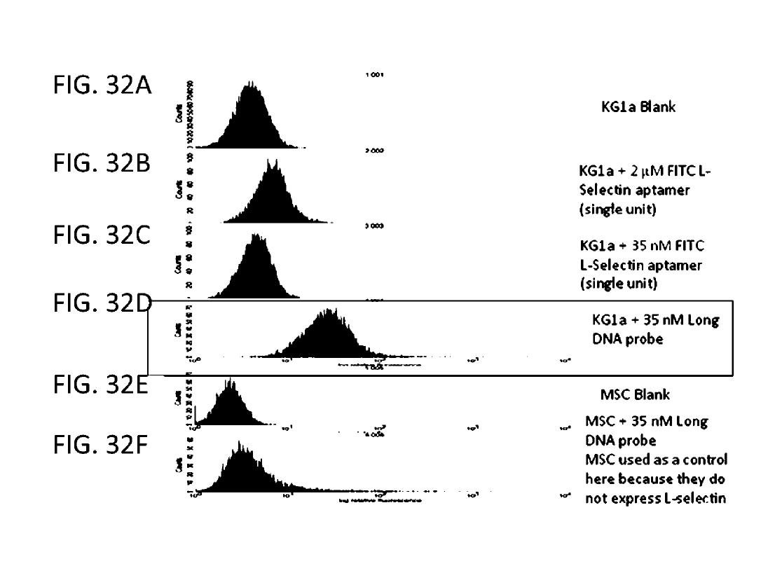

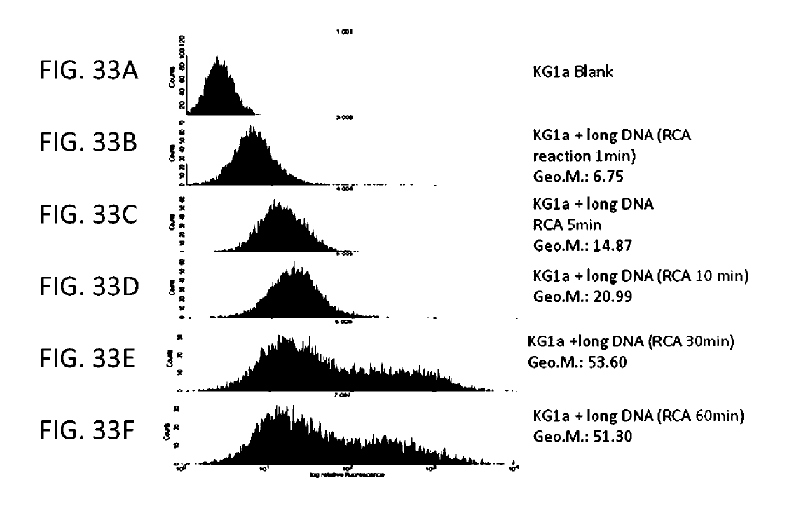

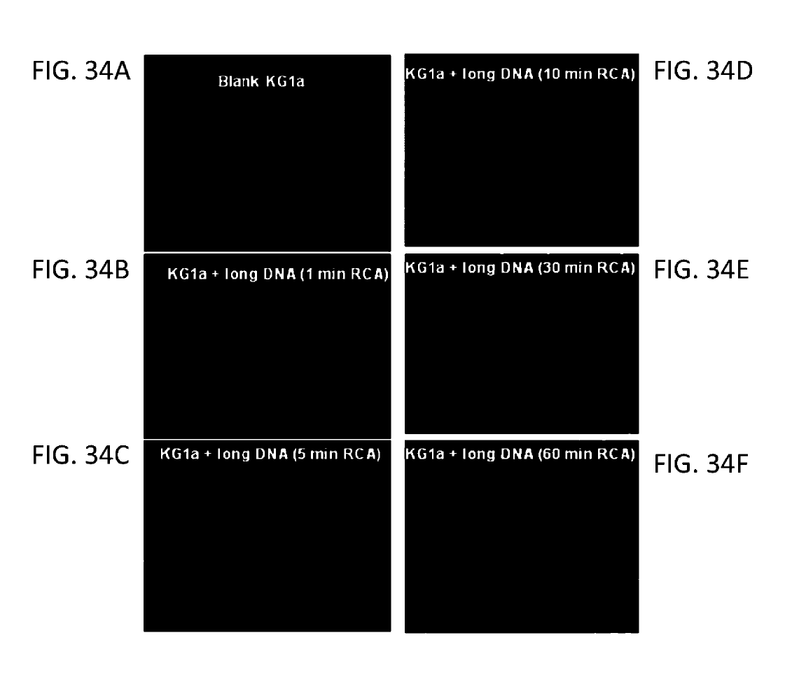

In the present disclosure, long DNA molecules can be produced by RCA on cell surface in situ (see FIG. 30A). An aptamer domain that specifically recognizes target surface markers is coupled with RCA primer and circular template. After incubation with cells, the RCA primer/circular template becomes attached onto cells via aptamer/target interactions. Subsequently, DNA polymerase (e.g., phi29 DNA polymerase) is added and initializes the RCA reaction in the presence of dNTPs (dATP, dTTP, dCTP and dGTP) to produce long (e.g., micron-long) DNA molecules. These long DNA molecules contain repeating units and can be labeled by dyes attached to complementary DNA strands. The labeled cells can then be analyzed by flow cytometry and microscopy. The present disclosure includes, but not limited assays for ultrasensitive detection of targets on cells (e.g., L-selectin on KG1a cells). These long DNA molecules can contain DNA enzyme strands which can convert chromogenic reagents into color signals and can be recorded by standard plate reader.

In the present disclosure, long DNA molecules can be produced (and, e.g., dye labeled) in solution first, and then used to label cell and detect cell surface markers (see FIG. 30B). The present disclosure includes use of such long DNA molecules for detection of targets on cells (e.g., for detection of L-selectin on KG1a cells).

In the present disclosure, the long DNA probes can also be produced on beads and nanoparticles to maximize the signal amplification (see FIG. 30C). One particle can include tens to hundreds of long DNA strands. Once one particle binds to cell surface markers, a single binding event can be amplified more than 10,000 times.

In the present disclosure, multiple different aptamer domains can be produced in the long DNA strands by encoding their respective complementary sequences in the circular templates. Therefore, this method can be used for multiplex assaying of numerous targets at the same time. In the present methods, when using dyes to label long DNA strands, multiple different dyes can be easily incorporated by designing different complementary strands, which makes multi-color detection feasible. In the present invention, the length of DNA molecules and therefore the numbers of labeled dyes can be easily adjusted by adjusting the RCA reaction time.

### Cell Targeting Methods

Further included in the present disclosure are methods of engineering cells with aptamers that can target or “home” cells to surfaces and other cells much like “adhesion molecules.” These aptamer-modified cells can adhere to and interact with, in a specific and fully controlled manner, surfaces and other cells that possess targets of the aptamers. In the present invention, aptamer-modified cells enable efficient cell targeting, homing, and engraftment to targeted tissues in cell therapy and regulation of desirable biological functions via promoted cell-cell interactions. Aptamers can be used to target cells to any desired cellular or extracellular location by targeting the cells to a particular molecule found in that location. In one embodiment, the present disclosure includes selectin aptamer-attached cells. Selectins, including L, P, and E-selectins, are crucial cell adhesion molecules that regulate cell rolling, adhesion, homing, cell-cell interactions at in many biological processes such as inflammation (Tedder et al., 1995, FASEB J., 9:866-873). The present disclosure includes L-selectin DNA aptamer-attached MSCs. The aptamers on the cell surface enable MSCs to tether strongly on L-selectin coated surfaces and L-selectin expressing cells, including leukocytes, under both static and flow conditions. In the present invention, modifying MSCs with aptamers that target leukocytes can be used to enhance MSC therapy, since it is known that MSC-Leukocyte cell-cell contact has added benefit for down-regulation of inflammation.

The present disclosure also includes methods of preparation of P-selectin aptamer attached cells. In some embodiments, P-selectin RNA aptamers can be attached to MSCs, which enables MSCs to tether strongly to P-selectin coated surfaces. The aptamer modified MSCs that specifically target P-selectin expressing cells are of particular importance for MSC-based therapy including tissue repair, regeneration at damaged tissues, and down-regulation of inflammation, as ECs transiently express P-selectin at sites of inflammation (Lawrence et al., 1991, Cell, 65:850-873; Ley et al., 2004, Bone Marrow Transplant., 33:597-604). In the present disclosure, P-selectin aptamer modified MSCs can be used to specifically and efficiently target such sites. In particular, aptamer modified MSCs that secrete paracrine factors can be targeted to damaged tissues to down regulate inflammation at sites of inflammation.

The aptamer-modified cells described herein can be used for promoting cell and surface/cell interactions for any given purpose. The methods present in the present invention, for promoting desirable cell-cell interactions in cell therapy, are suited for a variety of administration methods including local injection of the cells or by systemic infusion.

Cell-cell interactions are important for many biological processes. Promoting a cell-cell interaction, which does not exist otherwise, is of great therapeutic interest.

Leukocyte and hematopoietic stem cells (HSCs) can bind to activated ECs during inflammation (Lawrence et al., 1991, Cell, 65:850-873; Ley et al., 2004, Bone Marrow Transplant., 33:597-604). However, a major challenge in cell therapy, and MSC therapy in particular, is the inability to target the in vitro cultured cells to a desirable location (e.g., inflammation sites). For example, the homing efficiency of systemically infused MSCs to desired tissues is typically ≦1% (Kawada et al., 2004, Blood, 104:3581-87).

MSCs can regulate leukocyte functions via direct contact and released cytokines in solution (Nauta et al., 2007, Blood, 110:3499-3506). Direct MSC/leukocyte interactions, in a close proximity, can be beneficial especially when paracrine factors released from MSCs would otherwise diffuse into bulk spaces and become too dilute before reaching the inflammatory cells.

### Cell Administration

A variety of means for administering cells to subjects are known to those of skill in the art, and can be used in the present methods. Such methods can include systemic injection, for example i.v. injection or implantation of cells into a target site in a subject. Other methods can include intratracheal delivery, intrathecal delivery, intraosseous delivery, pulmonary delivery, buccal delivery, and oral delivery. Cells can be inserted into a delivery device which facilitates introduction by injection or implantation into the subjects. Such delivery devices can include tubes, e.g., catheters, for injecting cells and fluids into the body of a recipient subject. In one preferred embodiment, the tubes additionally have a needle, e.g., a syringe, through which the cells of the invention can be introduced into the subject at a desired location. In some embodiments, cryopreserved cells are thawed prior to administration to a subject.

As used herein, a “subject” or a “patient” refers to any mammal (e.g., a human), such as a mammal that can be susceptible to a disease. Examples include a human, a non-human primate, a cow, a horse, a pig, a sheep, a goat, a dog, a cat, or a rodent such as a mouse, a rat, a hamster, or a guinea pig. A subject can be a subject diagnosed with the disease or otherwise known to have the disease. In some embodiments, a subject can be diagnosed as, or known to be, at risk of developing a disease. In certain embodiments, a subject can be selected for treatment on the basis of a known disease in the subject. In some embodiments, a subject can be selected for treatment on the basis of a suspected disease in the subject. In some embodiments, a disease can be diagnosed by detecting a mutation associate in a biological sample (e.g., urine, sputum, whole blood, serum, stool, etc., or any combination thereof. Accordingly, a compound or composition of the invention can be administered to a subject based, at least in part, on the fact that a mutation is detected in at least one sample (e.g., biopsy sample or any other biological sample) obtained from the subject. In some embodiments, a cancer can not have been detected or located in the subject, but the presence of a mutation associated with a cancer in at least one biological sample can be sufficient to prescribe or administer one or more compositions of the invention to the subject. In some embodiments, the composition can be administered to prevent the development of a disease such as cancer. However, in some embodiments, the presence of an existing disease can be suspected, but not yet identified, and a composition of the invention can be administered to prevent further growth or development of the disease.

The cells can be prepared for delivery in a variety of different forms. For example, the cells can be suspended in a solution or gel or embedded in a support matrix when contained in such a delivery device. Cells can be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating cells as described herein in a pharmaceutically acceptable carrier or diluent and, as required, other ingredients enumerated above, followed by filtered sterilization.

It is preferred that the mode of cell administration is relatively non-invasive, for example by intravenous injection, pulmonary delivery through inhalation, oral delivery, buccal, rectal, vaginal, topical, or intranasal administration. However, the route of cell administration will depend on the tissue to be treated and can include implantation. Methods for cell delivery are known to those of skill in the art and can be extrapolated by one skilled in the art of medicine for use with the methods and compositions described herein.

Direct injection techniques for cell administration can also be used to stimulate transmigration through the entire vasculature, or to the vasculature of a particular organ, such as for example liver, or kidney or any other organ. This includes non-specific targeting of the vasculature. One can target any organ by selecting a specific injection site, such as e.g., a liver portal vein. Alternatively, the injection can be performed systemically into any vein in the body. This method is useful for enhancing stem cell numbers in aging patients. In addition, the cells can function to populate vacant stem cell niches or create new stem cells to replenish the organ, thus improving organ function. For example, cells can take up pericyte locations within the vasculature.

In some embodiments, the cells are introduced into the subject as part of a cell aggregate (e.g., a pancreatic islet), tissue, or organ, e.g., as part of an organ transplant method.

Delivery of cells can also be used to target sites of active angiogenesis. For example, delivery of endothelial progenitor cells or mesenchymal stem or progenitor cells can enhance the angiogenic response at a wound site. Targeting of angiogenesis can also be useful for using cells as a vehicle to target drugs to tumors.

If so desired, a mammal or subject can be pre-treated or co-treated with an agent. For example, an agent is administered to enhance cell targeting to a tissue (e.g., a homing factor) and can be placed at that site to encourage cells to target the desired tissue. For example, direct injection of homing factors into a tissue can be performed prior to systemic delivery of ligand-targeted cells. In some embodiments, an agent can be administered to enhance permeation of cells to modulate the release of agents from inside to outside the cell. Exemplary permeation enhancers include dendrimers, cell-penetrating peptides, and cationic polymers. In some embodiments, the cells are provided in a delivery device (e.g., an encapsulating material such as a hydrogel) and the agent is also present in the delivery device.

### EXAMPLES

### Example 1

### Fluorescence Quenching PDGF Aptamer Sensors on Beads

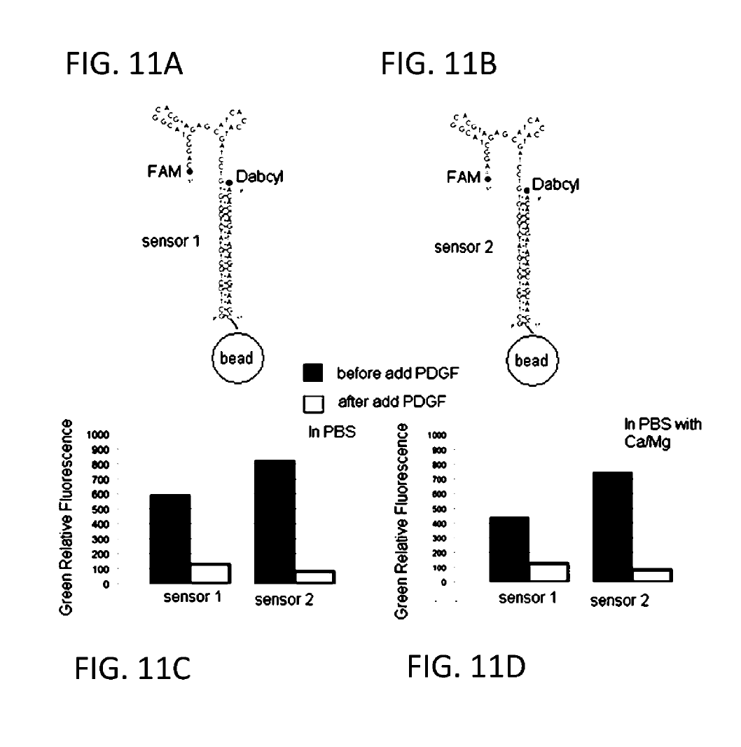

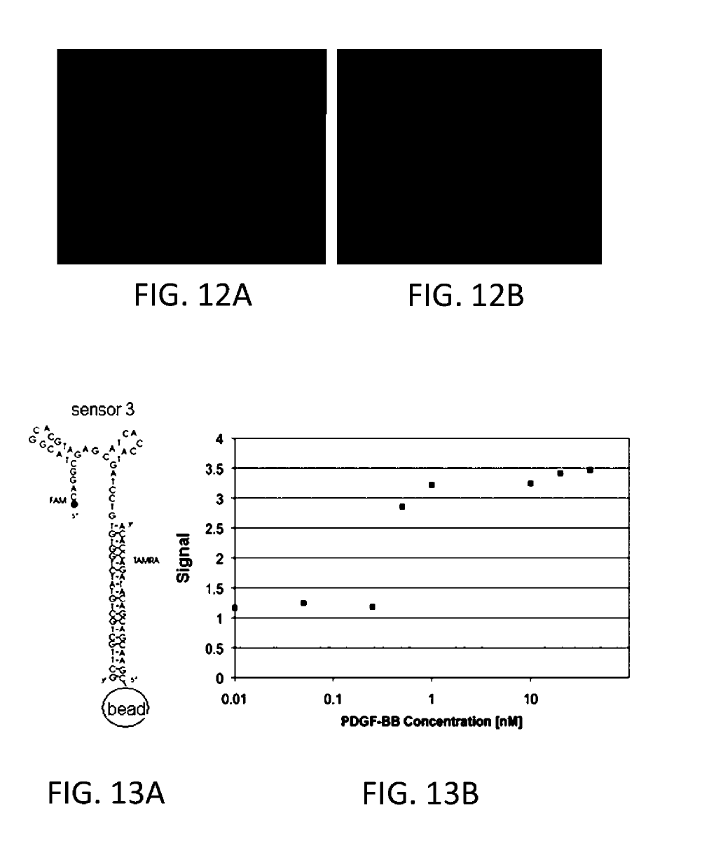

A PDGF aptamer sensor was synthesized with FAM at the 5′ end of the aptamer and a quencher, dabcyl, at the 3′ end of the complementary strand, with a biotin molecule attached at the other end (FIG. 11A). Upon binding to the target PDGF, the aptamer undergoes a conformational change which brings FAM and dabcyl closer to each other and the fluorescence of FAM is quenched. One base pair C-G of the aptamer sensor was eliminated by changing a C base to A base (FIG. 11B). Therefore, sensor 2 has less nonspecific folding and better performance in the presence of divalent metal ions than sensor 1. Sensor 1 and 2 were immobilized on streptavidin beads, and their performances in PBS (FIG. 11C) and PBS with Ca/Mg (FIG. 11D) were studied by flow cytometry and microscopy (FIGS. 12A-12B). Upon adding PDGF (10 nM), the FAM green signal was quenched. Sensor 2 functioned better than sensor 1 in PBS with and without Ca/Mg.

The signal of the sensor on streptavidin bead was PDGF concentration dependent. In a further experiment, quenching Sensor 3 was synthesized, where FAM and TAMRA were used as FRET donor dye and acceptor dye, respectively (FIG. 13A). Upon adding PDGF, the aptamer folds and brings two dyes into close proximity where FAM fluorescence signal is quenched by TAMRA. The amount of fluorescence quenching was analyzed by flow cytometry and plotted as signal in the Y axis (FIG. 13B).

### Example 2

### Fluorescence Quenching PDGF Aptamer Sensors on Cells

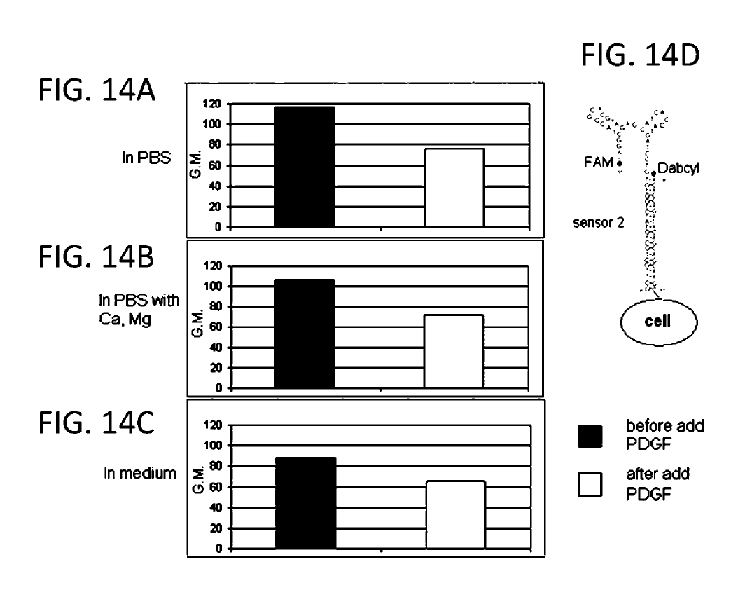

Quenching sensor 2 performance on MSCs (FIG. 14D) was studied by flow cytometry in PBS (FIG. 14A), PBS with Ca/Mg (FIG. 14B) and medium (FIG. 14C). Medium contained 5% FBS, 1% (v/v) L-Glutamine, 1% (v/v) Penn-Strep, and α-MEM. Upon addition of PDGF (10 nM), the FAM green signal was quenched. The performance of the sensor was best in PBS, followed by in PBS with Ca/Mg, and then in medium.

For cell surface modification, MSCs (˜1M after trypsinization) were dispersed in Biotin-NHS solution (1 mM in PBS, 1 mL) and the solution was allowed to incubate for 10 minutes at room temperature. After washing, streptavidin solution (50 μg/mL in PBS, 1 mL) was then used to treat the cells for 5 minutes. Finally, biotin-modified sensor solution was added, and the suspension was incubated for 5 minutes at room temperature. The cells were then washed once by PBS and subsequently used for experimentation.

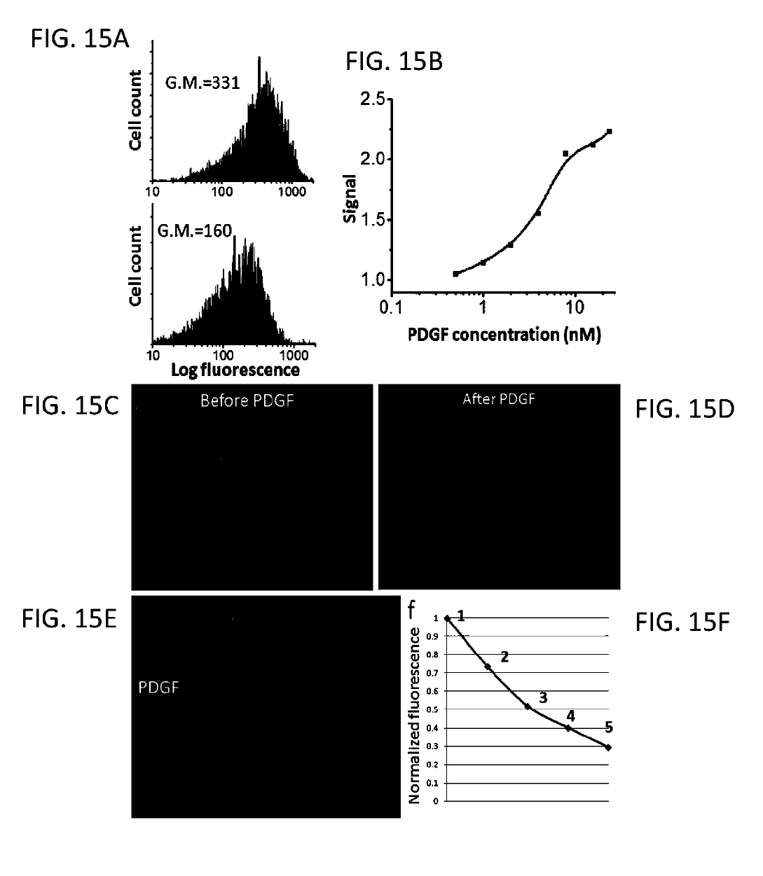

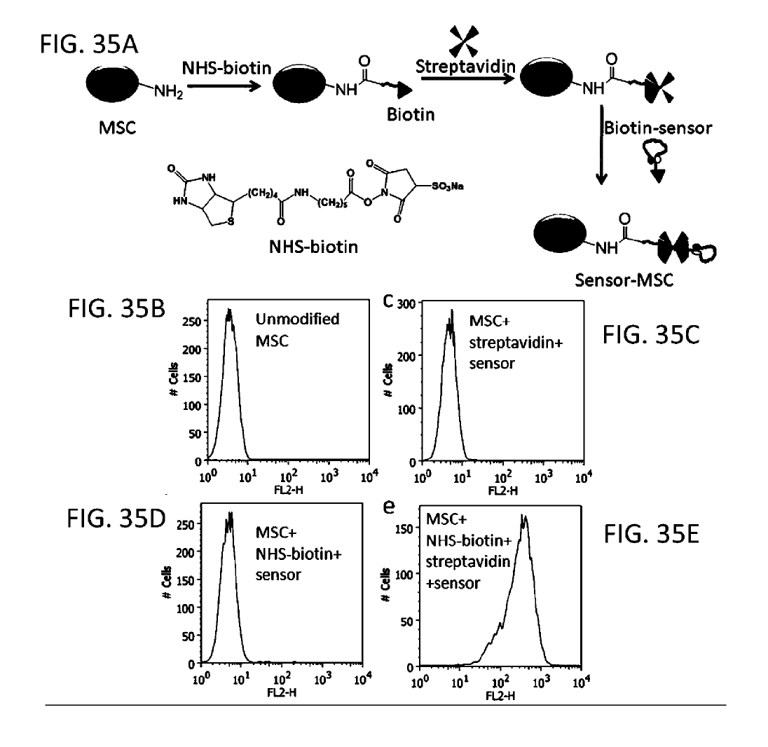

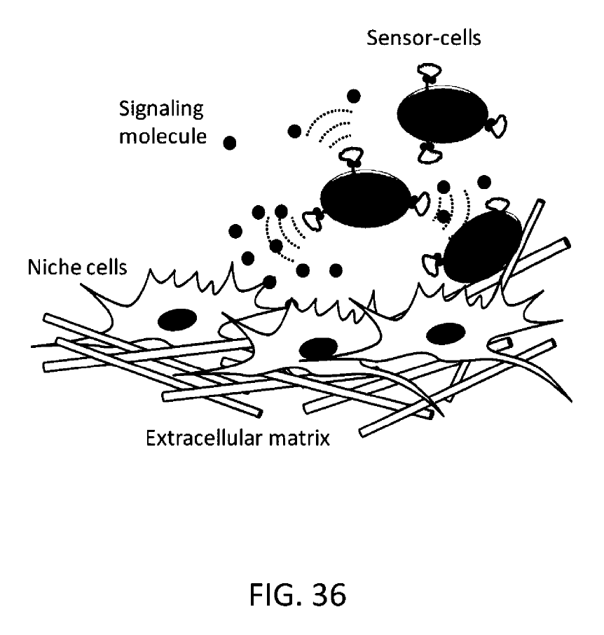

A significant advantage of the sensor-cell platform described herein is that it uses simple chemistry to attach sensors on the cell membrane that bypasses the complexity of genetic, enzymatic or metabolic engineering approaches used previously for cell surface engineering. This allows the attachment multiple types of sensors simultaneously. Specifically, as shown in FIG. 35A, our generic cell modification procedure consists of three steps as we have previously described (Sarkar et al., 2003, Bioconj. Chem. 19:2105-09). Briefly: 1) cell biotinylation by treating cell surface amines with sulfonated biotinyl-N-hydroxy-succinimide (NHS-Biotin), 2) binding with streptavidin, and 3) attachment of biotinylated aptamer sensors. This versatile chemical approach enables one to easily tune the site density of attached molecules on each cell by, for example, adjusting reagent concentration and reaction times during the conjugation. In a typical reaction, we have determined that ˜21,000 molecules are attached on each MSC. Using this procedure, we have recently demonstrated that MSCs modified with a cell rolling ligand (Sialyl Lewis X, SLeX) show robust rolling performance on both P-selectin-coated substrates in vitro and on activated endothelial cells in vivo. Such surface functionalization chemistry does not impact important cell phenotype (i.e. viability, adhesion, proliferation, secretion of paracrine factors and multilineage differentiation) nor their homing ability or transendothelial migration to an inflamed site following systemic infusion (Sarkar et al., 2003, Bioconj. Chem. 19:2105-09; Sarkar et al., 2010, Biomaterials, 31:5266-74). In this study, using the same approach, we have successfully immobilized biotin-modified aptamer sensors on the MSC surface as evidenced by a fluorescent signal on cells following modification (see FIGS. 35B-35E). The sensor-modified cells are sensitive to PDGF (FIG. 15A).

Using the quench sensor (FIG. 14D) as an example, we have demonstrated sensor performance on the cell surface as evidenced by the decrease of fluorescence upon addition of PDGF. Sensors on the cell surface respond to PDGF instantaneously (within seconds) (data not shown). The sensor signal on the cell surface, measured immediately after mixing with PDGF, quantitatively correlates with the concentration of PDGF added into the cell solution in PBS (FIG. 15B). Moreover, the detection range of sensors on the cell surface is from several hundred pM to low nM. This is in the range of serum PDGF concentration which is 400-700 μM under physiological conditions or higher under pathological conditions (e.g., within tumors). The sensor performance was also monitored by fluorescent microscopy before (FIG. 15C) and immediately after addition of 10 nM PDGF in PBS (FIG. 15D). In another experiment, PDGF (2 μM) was added from the top/left corner (arrow indicates PDGF flow direction) by a pipette tip to sensor modified MSCs and image was immediately recorded (FIG. 15E). The PDGF gradient was separated into five regions using image analysis and the fluorescent intensity of 10 representative cells from each region were averaged and plotted in FIG. 15F. The sensor signal in region 1 is defined as 1 and other regions were normalized accordingly.

This example demonstrates that nucleic acid sensors on cells can be used to detect the presence of targets in the cellular environment.

### Example 3

### Tuning of Sensor FRET Parameters

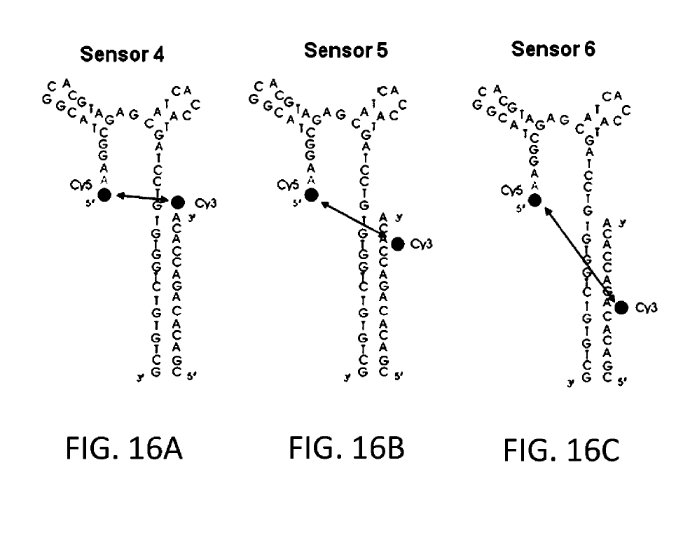

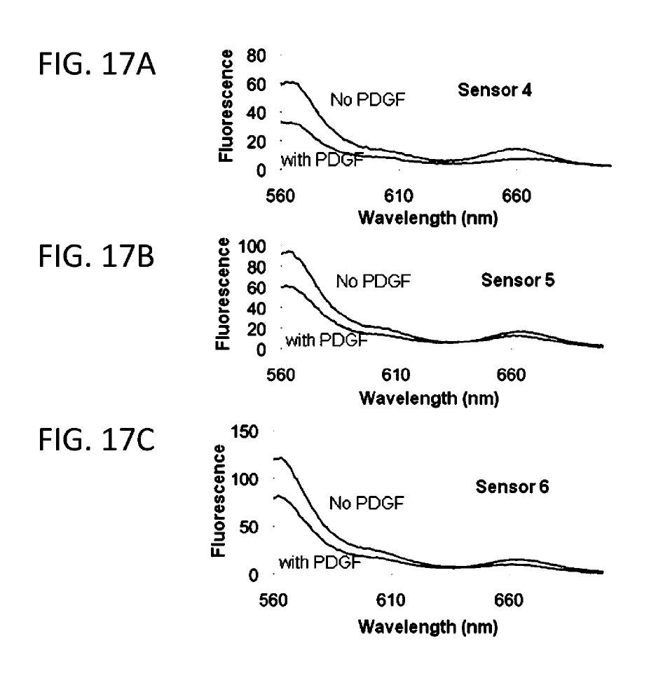

FRET PDGF sensors 4, 5, and 6 were synthesized using Cy3 and Cy5 as FRET donor and receptor, respectively (FIGS. 16A-16C). The distance between Cy3 and Cy5 in the sensors followed sensor 6>sensor 5>sensor 4. The performance of sensors 4, 5, and 6 (10 nM) in PBS solution was recorded by fluorometer in the absence and presence of 10 nM PDGF. In sensor 4, upon adding PDGF (10 nM), both Cy3 and Cy5 dyes are quenched. In sensors 5 and 6, Cy3 and Cy5 were quenched and enhanced, respectively upon adding PDGF (10 nM). This example demonstrates the tunability of FRET performance by placing two dyes at different positions on sensor constructs.

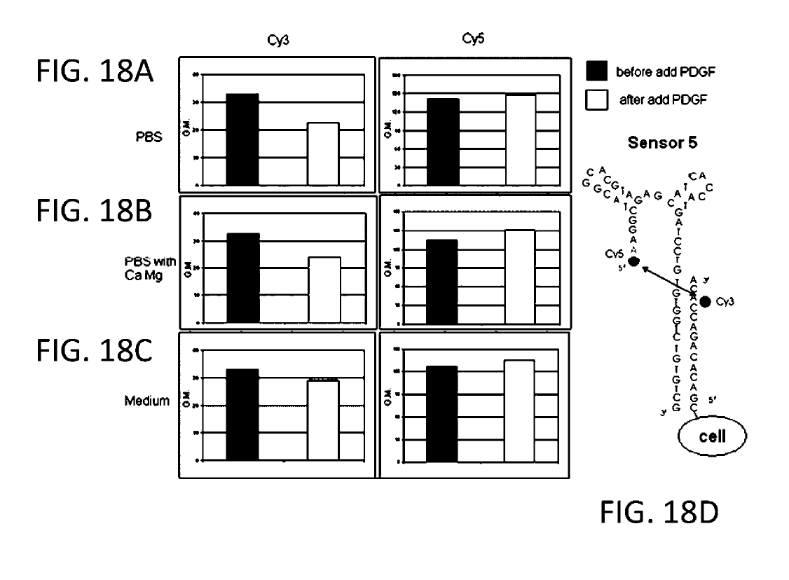

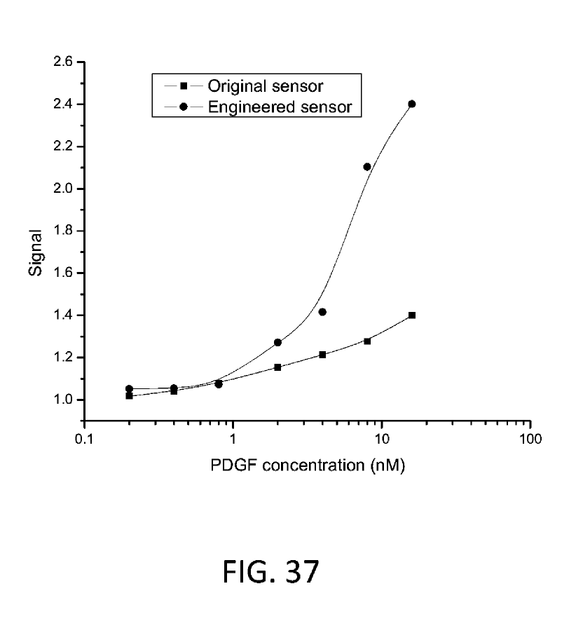

The performance of Cy3-Cy5 FRET sensor 5 on MSCs (FIG. 18D) in PBS, PBS with Ca/Mg and medium was determined. In all cases, upon addition of PDGF (10 nM), Cy3 fluorescence was quenched and Cy5 fluorescence increased (FIGS. 18A-18C). The performance of sensor 5 was the best in PBS, followed by in PBS with Ca/Mg and then in medium. The Cy3 and Cy5 fluorescence intensity in these data were G.M. from flow cytometry analysis. The ratio of fluorescence obtained by flow cytometry, before and after addition of PDGF, is shown in FIG. 37.

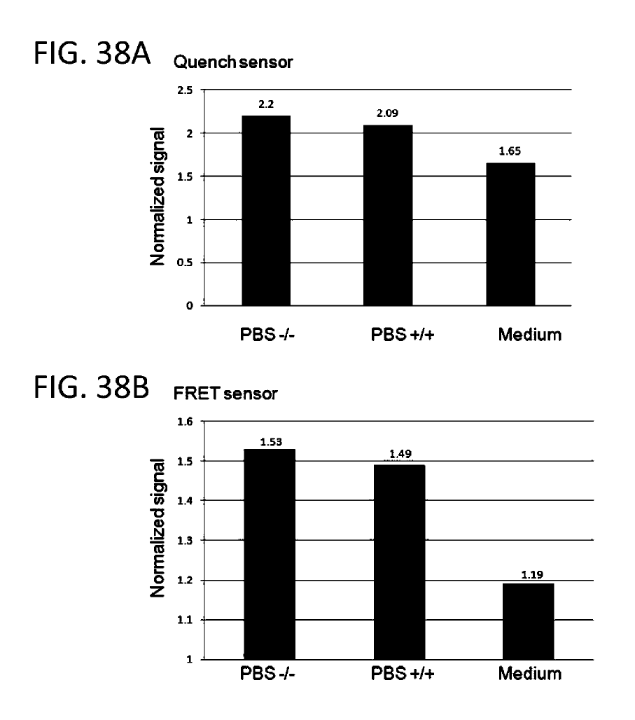

Ratios of fluorescence for engineered aptamer sensors on cells are also presented in FIGS. 38A-38B.

### Example 4

### Thrombin Sensor

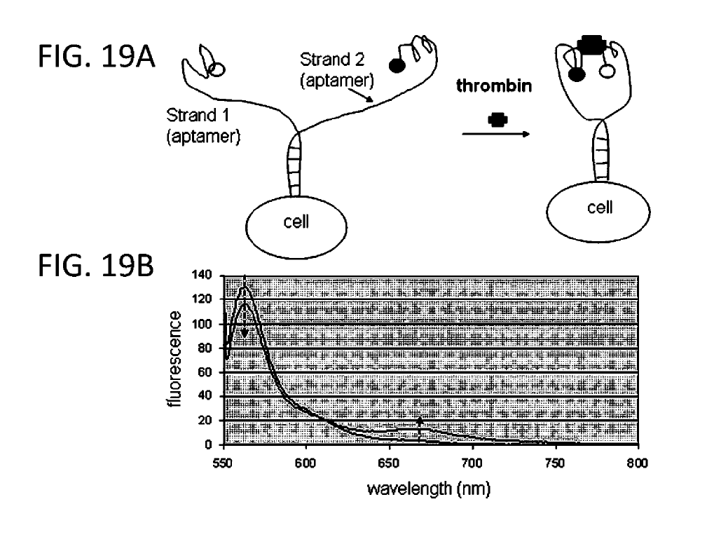

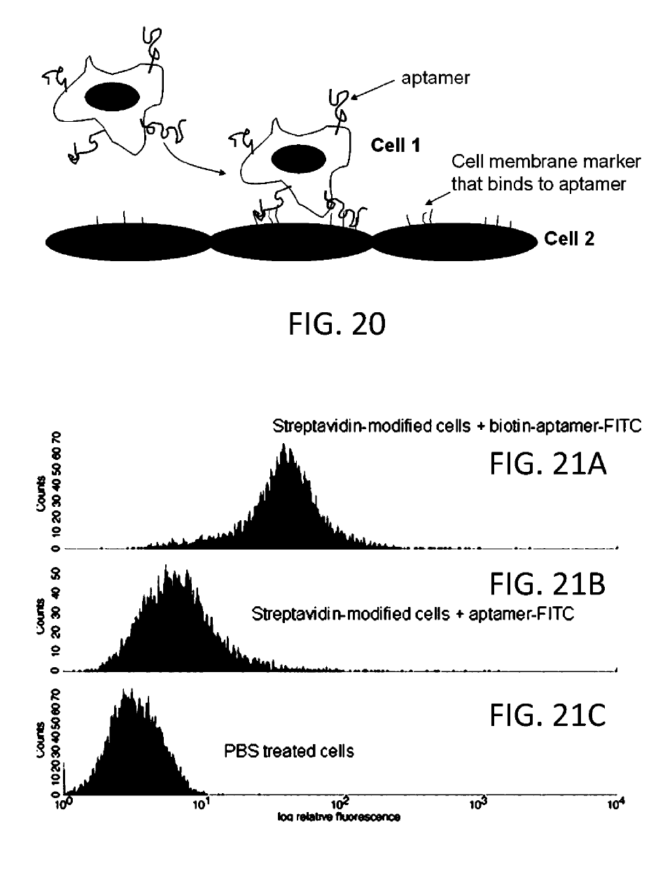

A thrombin sensor was synthesized with two aptamer strands attached to counterpart FRET dyes on cells (FIG. 19A). Upon binding to thrombin, these two stands of aptamer bring the attached FRET dyes into close proximity which gives a FRET signal. FIG. 19B shows fluorescence spectra of thrombin sensor (10 nM) in PBS with Ca/Mg before and after addition thrombin (5 NIH units/500 μL).

### Example 5

### Spatial-Temporal Sensing of PDGF at Single Cell Resolution

To determine whether sensors on cells produce a fluorescence signal in real-time that can be resolved with high spatial resolution at a single cell level, PDGF was added in close proximity to the cells at a constant flow rate though a micromanipulator-mounted microneedle coupled to a microinjector operated at constant pressure. Microneedle experiments were performed using a microinjector (FemtoJet, Eppendorf) with Eppendorf Femtotips and an Eppendorf micromanipulator (InjectMan NI 2, Eppendorf). Glass microneedles with inner tip diameters of ˜3 μm were made using a micropipette puller (P-97 Sutter Instrument Company). Microneedles were backfilled with the PDGF-BB (2 μM in PBS) using Eppendorf Femtotips Capillary Pipet Tips Microloaders. The microneedle, controlled by a micromanipulator, was lowered onto a dish with sensor-engineered MSCs settled on the surface in PBS, positioned at a defined lateral distance (˜40 μm) from the settled cells and approximately at a height of 30 μm from the underlying substrate. PDGF was released from the micropipette by applying a defined pressure (26 hectopascals). Simultaneously, phase contrast and fluorescence images of the cells were collected sequentially with a 1 second interval exposure time.

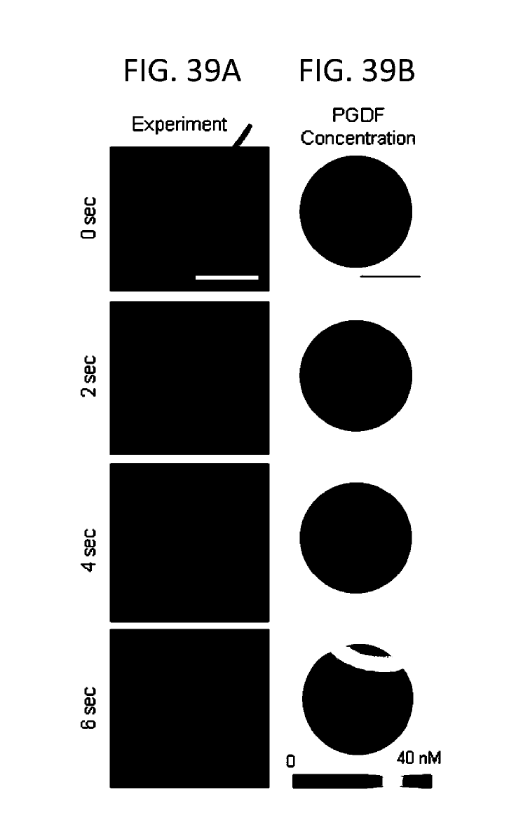

Fluorescence imaging showed spatial variation of the signal intensity over the cell's surface, which evolved over time as more PDGF was transported by the impinging flow to the cell surface (FIG. 39A). We also simulated the evolution of PDGF concentration in the vicinity of a cell using a three-dimensional unsteady convection-diffusion mass transport model. We used a finite volume scheme on a computational domain similar to the experimental setup using the commercial package FLUENT as described below. The evolution of concentrations on the surface of the cell was consistent with the observed fluorescence quenching behavior: The model predicted a transition of the PDGF concentration in the vicinity of the cell from 0 nM at t=0 to 40 nM at t=6 s (FIG. 39B). Given that this aptamer sensor when attached on the cell surface detects PDGF in the range of approximately 1-10 nM, the timescale of a cell response is consistent with the timescale required for the PDGF concentration to change, as predicted by the model. This agreement between the fluorescence response and the results of the model suggest that the aptamer sensor-modified cell indeed responds rapidly to changes in the PDGF concentration in the vicinity of the cell.

We built a computational model to estimate the local concentration of the PGDF near the cell. The following simplifying assumptions were made:
1. The flow is determined primarily by the direction and magnitude of injection velocity, and pipette body has minimal effect on the flow profile. This allows us to model the pipette as a thin vertical tube (Figure S8) and the direction of injection (30°) and magnitude of velocity (100 μm/s) are similar to those used in the experiment.
2. The flow is assumed to be symmetric about the pipette (one vertical plane of symmetry) allowing us to model only half of the computational domain.
3. It is assumed that the cells do not alter the flow pattern appreciably. Thus, we model only one cell (the cell of interest, which was photographed in the micro needle experiment), as a hemispherical cap, in our computational domain.
4. The cell surface concentration of aptamer was assumed to be low such that binding of PGDF on the surface does not appreciable alter the local PDGF concentration.

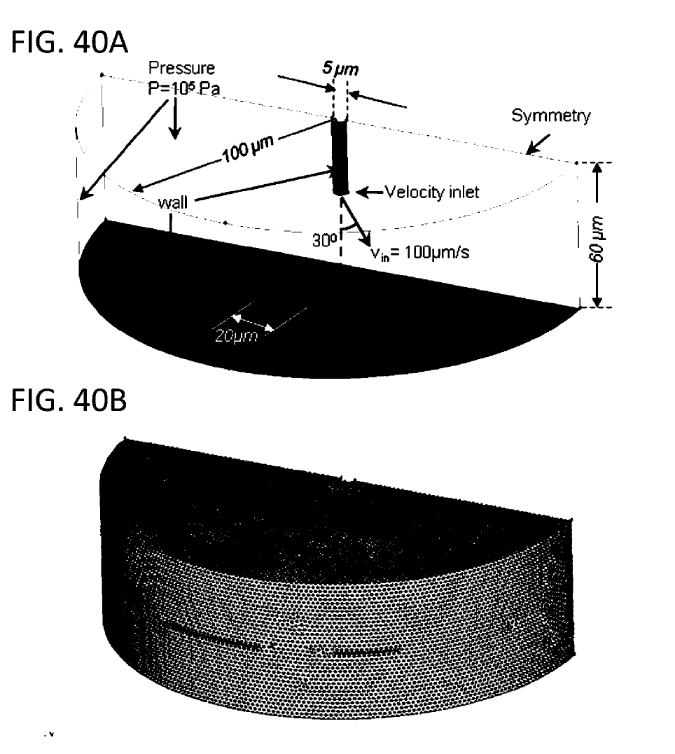

The computational domain was created and meshed in the commercial software GAMBIT (preprocessor of FLUENT, Ansys Inc.) using tetrahedral elements with edge lengths graded from 1 μm (boundary elements) to 3 μm (elements in the bulk fluid) (FIG. 40A). The meshed volume was exported into the computational software FLUENT (Ansys, Inc.) and the appropriate boundary conditions were applied (FIG. 40B). An unsteady incompressible laminar fluid flow model along with non-reacting species transport was chosen. This model uses finite volume method to discretize the continuity, Navier-Stokes and the mass transport equations shown below (gravity was neglected):

$$∇ • ( ρ \vec{\partial } ) = 0$$

$$\frac{\partial }{\partial t} ( ρ \vec{\partial } ) + \vec{\partial } • ∇ ( ρ \vec{\partial } ) = - ∇ P + μ {∇}^{2} \vec{\partial } \\ \frac{\partial C}{\partial t} + \vec{\partial } • ∇ C = D {∇}^{2} C$$

where ρ is the fluid density, \vec{θ} is the fluid velocity vector in cartesian coordinates, P is the static pressure, μ is fluid viscosity, C is the concentration of the transported species and D is the coefficient of diffusion of the species in the medium. The material properties used in our simulations were: ρ=998.2 kg/m³, μ=0.001003 kg/m-s, molecular weight of water=18.01 Da, molecular weight of PGDF-BB=24.3 kDa, D=1×10⁻¹° m²/s⁴. A segregated solver along with 1^{st} order implicit time stepping method was used. The pressure was discretized using PRESTO scheme while the momentum and species equation used a 2^{nd} order upwind scheme (both are inbuilt options in the software). The injected stream is assumed to have a PGDF mass fraction of 1 (accordingly, the calculated mass fraction of PDGF is interpreted as the concentration relative to the injected value). Unsteady simulations were performed with time step of 0.1 s with a maximum of 50 iterations per time step. The solution was terminated when all the residuals were below 10⁻⁴. The initial condition was no flow, and no PGDF present in the geometry (i.e. mass fraction of water is 1). The simulation was run in double precision mode.

### Example 6

### Cell Sensor Detects PDGF Produced by Neighboring Cells

The development of sensors that can be used to examine cell-cell communication in real-time can aid in elucidating mechanisms of intercellular communication. To test whether sensors immobilized on the cell surface can sense PDGF released from a neighboring cell in real-time, we utilized a microwell assay (Ogunniyi et al., 2009, Nature Protocol, 4:767-782) to study cell-cell signaling at a single cell level. Specifically, on a polymeric substrate containing an array of microwells (50 μm×50 μm×50 μm) that was made by soft lithography, we added a suspension of sensor-modified MSCs and PDGF producing cells (human breast cancer cell, MDA-MB-231, genetically engineered to produce PDGF. Microwell arrays were prepared by injecting a silicone elastomer mixture (polydimethylsiloxane (PDMS), Dow Corning Inc.) into a mold and curing at 70° C. for 2 h. The prepared arrays were 1 mm thick and bound to a glass slide. Each array consisted of 85,000 microwells (each 50 μm×50 μm×50 μm) arranged in 7×7 blocks. Arrays were treated for 30 s in an oxygen plasma chamber (Harrick PDC-32G) to render the surface sterile and hydrophilic. A sensor-MSC suspension (1×10⁵ cells/ml) was then placed on the surface of the array and cells were permitted to settle into the microwells by gravity. After 2 minutes, excess cells were washed away with serum-free media. Next, PDGF producing MDA-MB-231 cells (1×10⁵ cells/ml) were loaded into the wells as described above. After a brief incubation at 37° C. with 5% CO₂ the array was delivered to the microscope for imaging. All images were acquired on an automated inverted fluorescence microscope (Zeiss Observer Z-1, Carl Zeiss Inc.) equipped with a stage incubator (PM S1) and incubation chamber for live-cell imaging (37° C., 5% CO₂). The arrays were mounted on the microscope with a coverslip placed on top of the array. Phase and fluorescence (GFP and Cy5) micrographs were collected every 3 min for 6 hr. A total of ˜3000 microwells were imaged at each time-point. A custom-written image analysis program was used to identify the location and fluorescent intensity of each cell in the microwell array (Giepmans et al., 2006, Science, 312:217-224). A MATLAB script was written to track the fluorescence signal intensity of each sensor-MSC over the 6 hour time course. The signal intensity of each sensor-MSC was normalized to the signal intensity at t=0 minutes to account for the baseline cell-to-cell variation in sensor-MSC intensity. Sensor-MSCs were divided into groups based on the number of PDGF producing MDA-MB-231 cells residing in the same microwell (0, 1, 2, or 3+ PDGF producing MDA-MB-231 cells). More than a hundred MSCs from each group were tracked. The fraction of sensor-MSCs in each group with a signal intensity less than 50% of the initial signal intensity was calculated at each time-point.

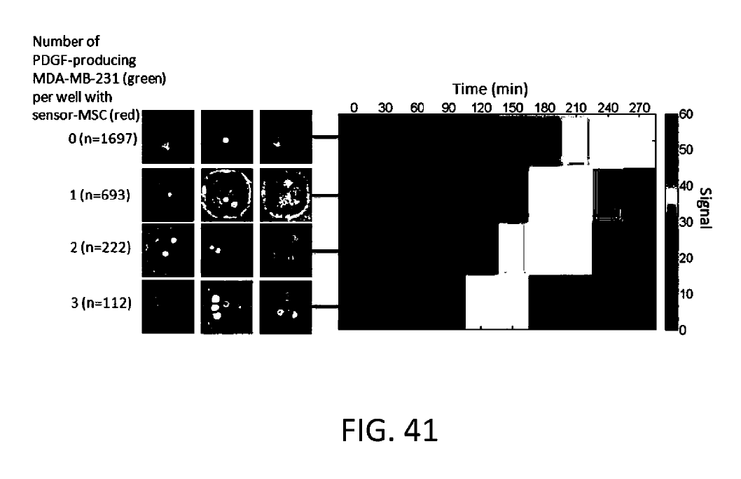

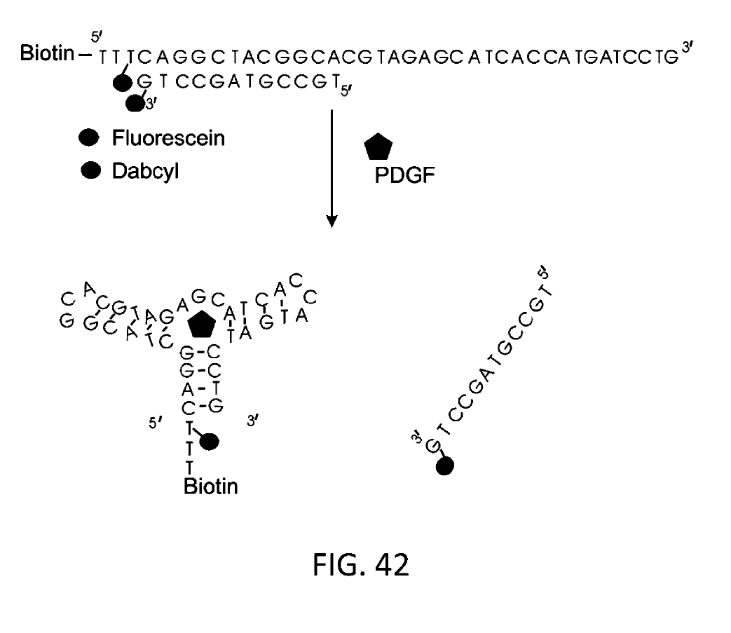

The production of PDGF was confirmed and quantified by ELISA. Cells settle by gravity into the microwells that contain subnanoliter volumes (0.1 nL) with different combinations of cell ratios (sensor-MSC:PDGF producing MDA-MB-231 cells=1:0, 1:1, 1:2, 1:3, FIG. 41). The fluorescence signal of sensor-MSCs was then imaged continuously over time (6 hours) as PDGF was produced by the MDA-MB-231 cell in the same microwell. As shown in FIG. 41, sensors on the MSC surface indeed produced a fluorescence signal which directly correlated with the number of MDA-MB-231 cells in the same microwell with sensor-MSC. By contrast, no significant signal difference in sensor signal was observed when sensor-MSCs were incubated alone or with native MDA-MB-231 cells (not engineered to secrete PDGF).

### Example 7

### Engineering Aptamer Ligands on the Surface of Mesenchymal Stem Cells

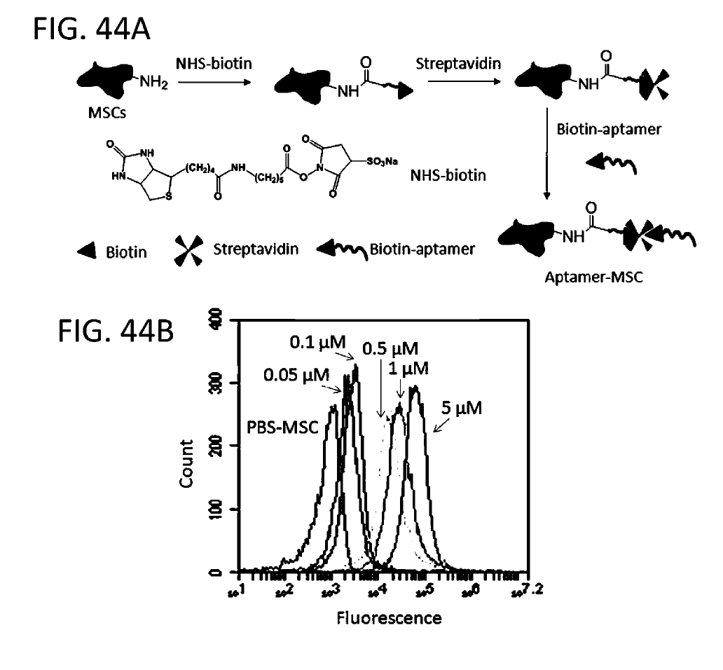

We conjugated aptamers to the MSC surface using a simple chemical approach. Specifically, the three step modification process includes 1) treatment of cells (in a suspension after trypsinization) with sulfonated biotinyl-N-hydroxy-succinimide (NHS-biotin) to introduce biotin groups on the cell surface, 2) complexing with streptavidin, and 3) coupling with biotinylated aptamers (FIG. 44A). It was found that typically ˜21,000 molecules were attached per MSC using this procedure. The successful conjugation of aptamers (conjugated with a fluorescent dye, FAM (a Fluorescein derivative); FAM-L-Aptamer-Biotin, 5′-FAM-tagccaaggtaaccagtacaaggtgctaaacgtaatggcttcggcttac-biotin-3′ (SEQ ID NO:7) on MSC was confirmed using flow cytometry (FIG. 44B). Importantly, the site density of aptamers on the cell surface could be readily tuned by adjusting the aptamer concentration used in the conjugation (FIG. 44B).

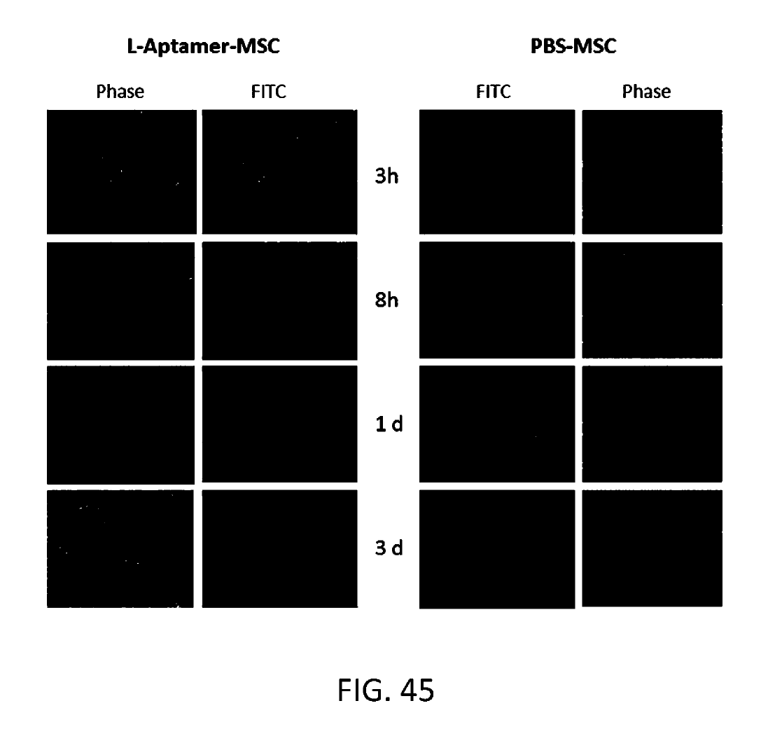

Given the potential for cell internalization and restriction enzyme degradation, we investigated the stability and accessibility of aptamers on the cell membrane under physiological conditions. We addressed this question by staining the L-selectin binding aptamer-modified MSC (L-Aptamer-MSC) at multiple time points after modification, with a complementary DNA conjugated to a dye (FAM) (FAM-Antisense, 5′-tacgtttagcaccttgtactggttacc-FAM-3′; SEQ ID NO:8) followed by fluorescent analysis. We confirmed that aptamers on the MSC surface were accessible to FAM-Antisense by flow cytometry immediately after modification. Modified cells in a 24 well plate were used to study the accessibility of cell bound aptamers through addition of the FAM at multiple time points and examination with fluorescence microscopy. Aptamers remained stable and accessible on the cell membrane for at least 24 hours in MSC cell culture medium at 37° C. (mimicking physiological conditions), as evidenced by strong positive fluorescent staining compared to the unmodified PBS-MSC controls (FIG. 45).

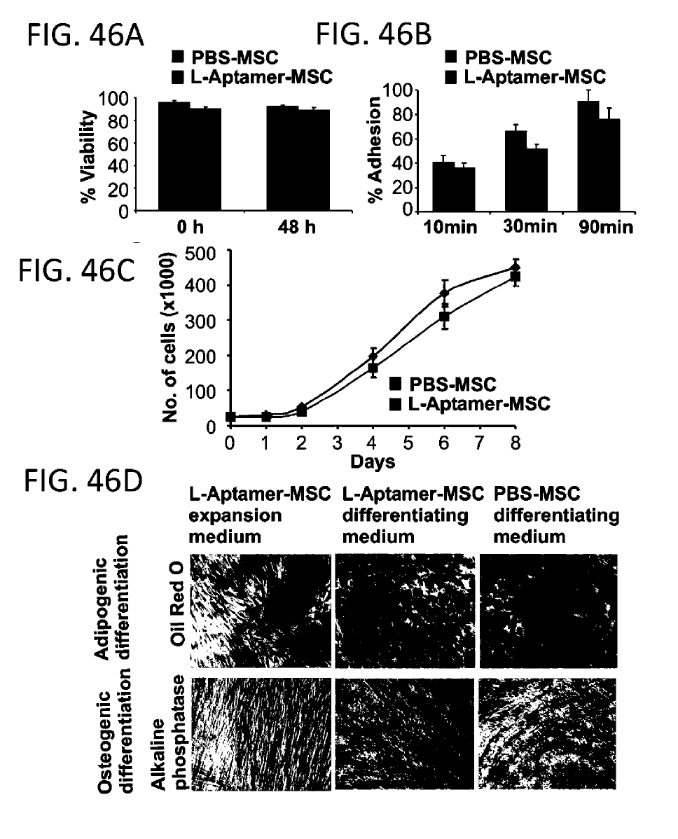

To examine the potential impact of aptamer conjugation on cell phenotype, we examined the viability, adhesion, proliferation and multilineage differentiation potential of L-Aptamer-MSC. The modification of MSCs with aptamers had minimal impact on MSC phenotype (FIGS. 46A-46D).

### Example 8

### L-Aptamer-MSCs Bind to L-selectin Coated Substrates Under Dynamic Flow Conditions

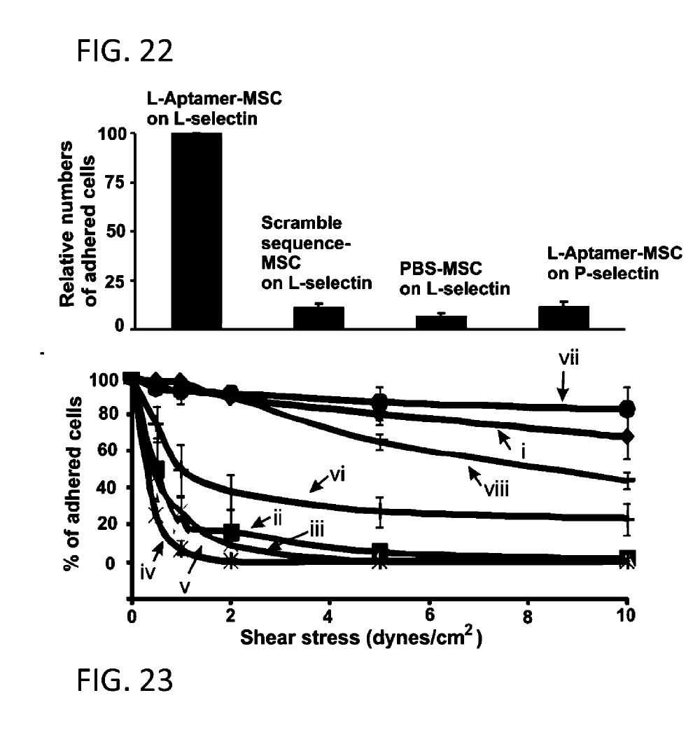

After we confirmed the successful conjugation and the availability of aptamers on the MSC surface, we investigated the interactions between L-Aptamer-MSCs and L-selectin coated surfaces under both static and flow conditions. For the static adhesion assay, aptamer modified and unmodified MSCs were incubated with L-selectin coated surfaces for 10 minutes, and unbound cells were then removed through rinsing. As shown in FIG. 22, the number of L-Aptamer-MSC that adhered to L-selectin surfaces (normalized to 100) was significantly higher than the control groups (8.95±2.23, 6.4±1.78, 9.6±2.3 for scrambled sequence aptamer modified MSCs on L-selectin, PBS MSC on L-selectin, and L-Aptamer-MSC on P-selectin, respectively).

We then investigated the adhesion of L-Aptamer-MSC on L-selectin coated surfaces under dynamic flow conditions using a parallel flow chamber. Specifically, cells were perfused into a flow chamber and then permitted to settle and interact with the substrate for 1 min before resuming flow conditions. The number of cells remaining on the surface was plotted as a percentage of the number of cells present before flow conditions were applied (Y axis) as a function of shear stress (X axis) (FIG. 23). L-Aptamer-MSC showed significantly stronger binding to the L-selectin coated surface than the controls. Controls included (i) PBS-MSC on L-selectin coated surfaces, (ii) L-Aptamer-MSC on P-selectin coated surfaces, (iii) L-Aptamer-MSC on L-selectin coated surfaces in the presence of 5 mM EDTA (EDTA removes divalent cations, e.g. Ca²⁺, that are essential for aptamer binding to L-selectin), (iv) scrambled sequence aptamer modified MSC on L-selectin coated surfaces, and (v) L-Aptamer-MSC on L-selectin coated surfaces blocked with L-selectin aptamers. Significantly, the ability of L-Aptamer-MSC to adhere to L-selectin coated surfaces was comparable to that of native HL-60 cells on L-selectin (line vii, FIG. 23). HL-60 cells can adhere strongly to L-selectin, up to shear stresses of 10 dyn/cm². Importantly, we can modulate the binding strength between L-Aptamer-MSC and L-selectin coated surfaces by simply titrating the aptamer site density on the MSC surface (i.e. by modulating the avidity). For instance, L-Aptamer-MSC with a lower site density of aptamer (prepared with 0.5 μM aptamer) showed significant but decreased binding to L-selectin coated surfaces (FIG. 23, line viii) compared to L-Aptamer-MSC prepared using 5 μM aptamer (FIG. 23, line i).

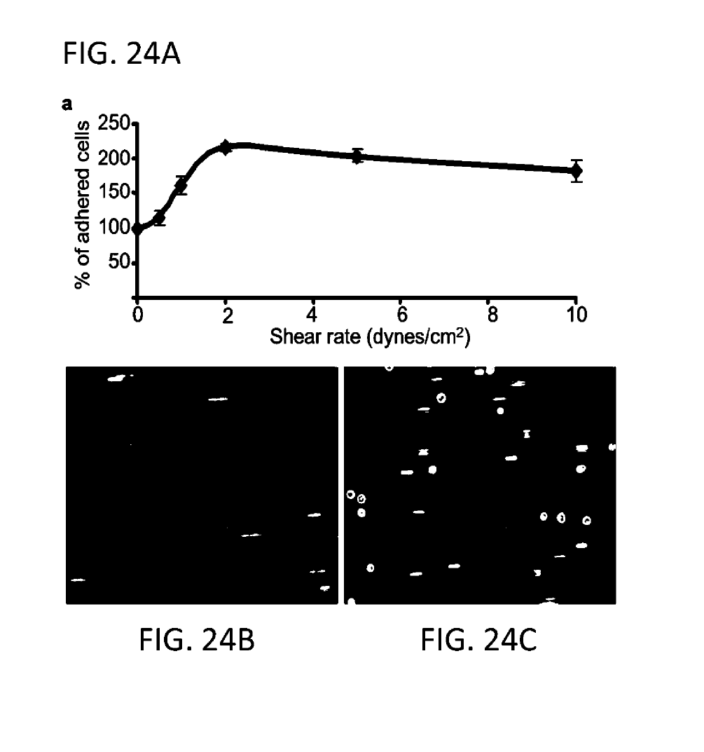

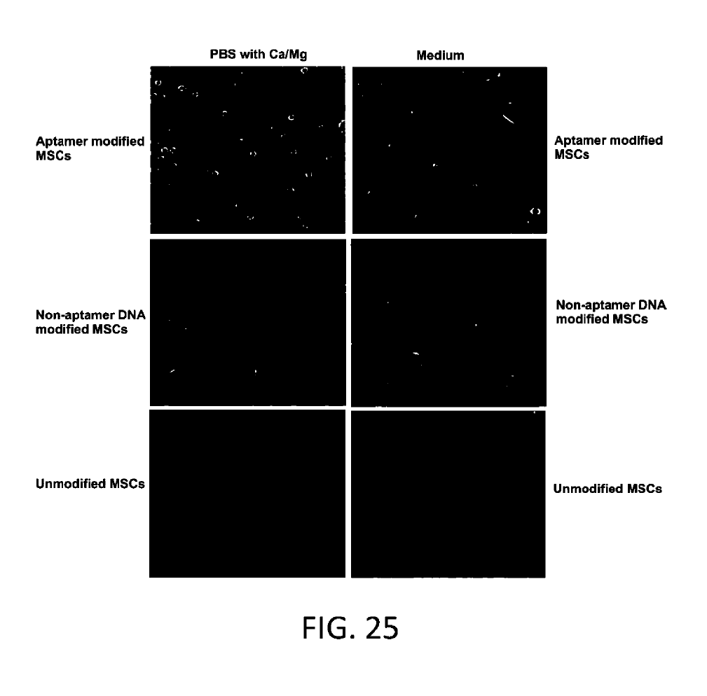

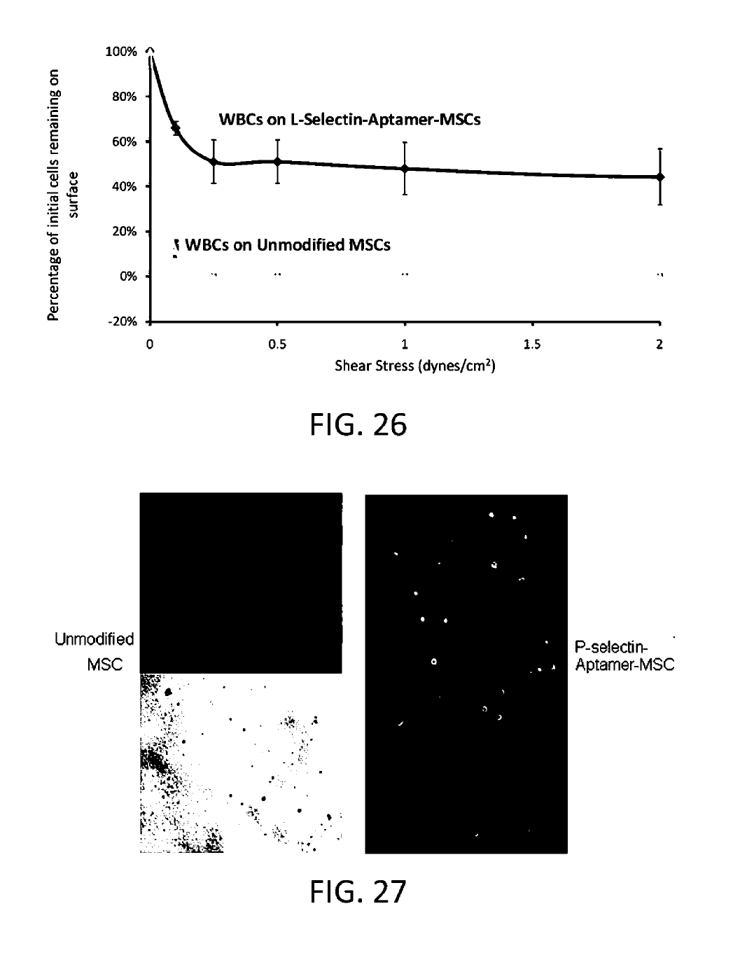

FIG. 23 shows the adhesion behavior of the cells that initially settled in the field of view before shear stress was applied (newly incoming cells which entered the field of view upon the application of shear flow were ignored). When newly incoming cells were considered, we observed a significant accumulation of L-Aptamer-MSC on L-selectin coated surface. As we increased shear stress, the total number of adhered cells in the field of view initially increased (up to 2 dyn/cm²) and then started decreasing at higher shear stresses (2-10 dyn/cm², FIG. 24A). Strikingly, L-Aptamer-MSC could be directly captured from the flowing cell suspension by L-selectin coated substrates under physiologically relevant flow conditions (up to 1.5 dyn/cm², FIGS. 24B, 24C). Cell-cell interactions leading to cell accumulation under dynamic flow conditions are critical in both normal physiology and in some cell-based therapies. For example, tethering of leukocytes or systemically infused therapeutic cells require contact and interaction with the endothelium under shear flow.

### Example 9

### P-Selectin Aptamer-MSCs Bind to P-Selectin Coated Surfaces

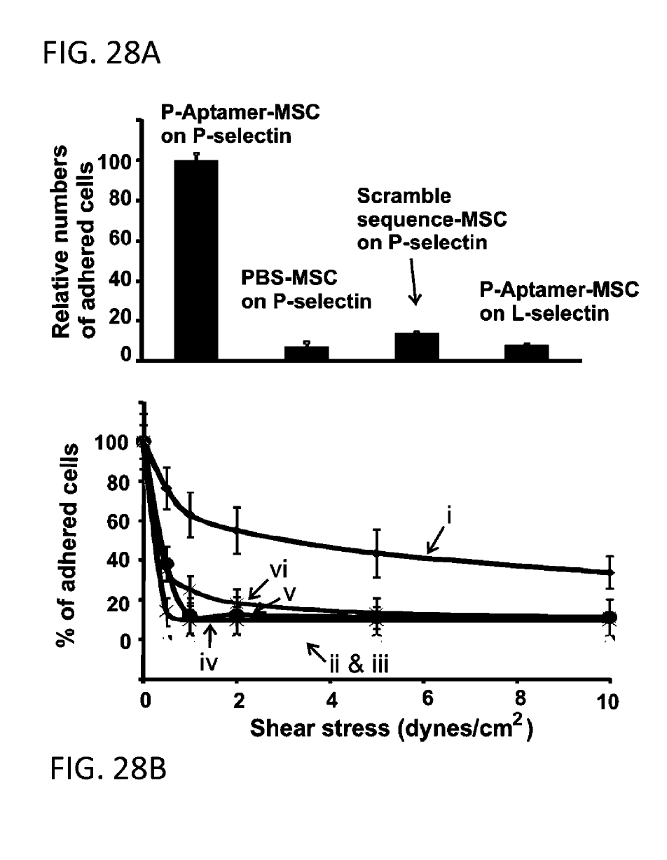

After establishing utility for the L-selectin binding DNA aptamer-MSC system, we then used the same procedure to conjugate P-selectin binding RNA aptamers onto MSC (P-Aptamer-MSC; 5′-biotin-cucaacgagccaggaacaucgacgucagcaaacgcgag-3′; SEQ ID NO:9) (C and U bases in this RNA molecule are modified with fluoro groups at 2′ to increase the RNA stability towards restriction enzyme digestion) and subsequently investigated their interactions with P-selectin coated surfaces under both static and flow conditions. As expected, cell surface tethered P-selectin aptamers facilitated the binding of MSC to P-selectin coated surfaces (FIG. 28A), which was otherwise absent under investigated conditions. Interestingly, the binding of P-Aptamer-MSC to P-selectin coated surfaces under flow conditions was not as effective as the L-Aptamer-MSC system: fewer cells remained adhered under high shear stress (FIG. 28B) and the tethering of cells on the substrate under continuous flow conditions was observed only up to 0.75 dyn/cm².

### Example 10

### Aptamer-Promoted Cell-Cell Interactions

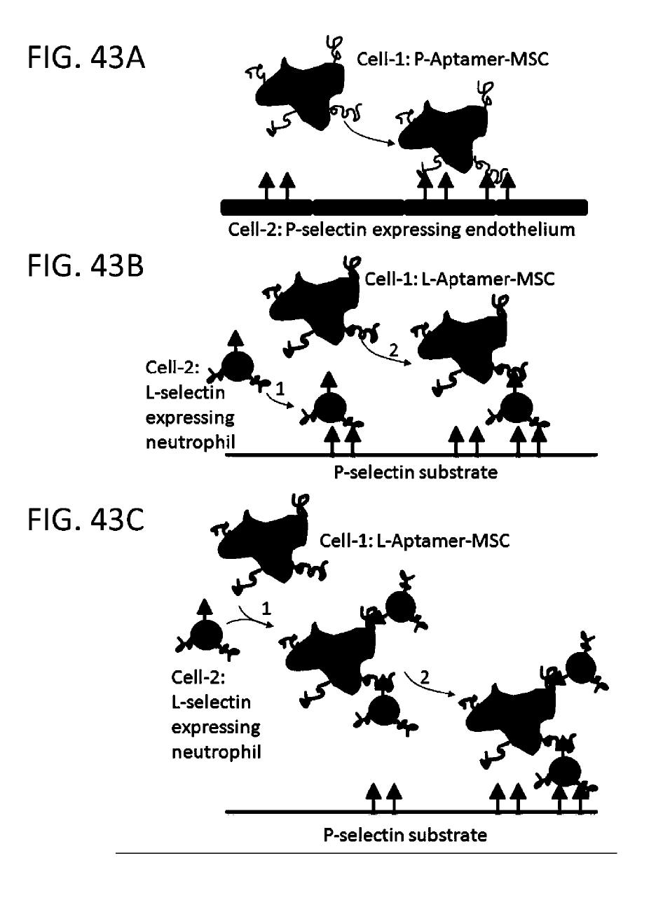

After demonstrating that aptamer-engineered MSC can bind specifically to selectin-coated substrates, we investigated aptamer promoted cell-cell interactions. We started with the first mechanism (FIG. 43A) to determine if the aptamer could promote a direct interaction between flowing MSC and adherent EC activated by inflammatory cytokines Human umbilical vein endothelial cells (HUVEC) were used as a model system, which are well known to express P-selectin when treated by inflammatory molecules such as histamine. In this study, we treated HUVEC with histamine for 10 min at 37° C. and confirmed the upregulation of P-selectin on HUVEC upon treatment using flow cytometry.

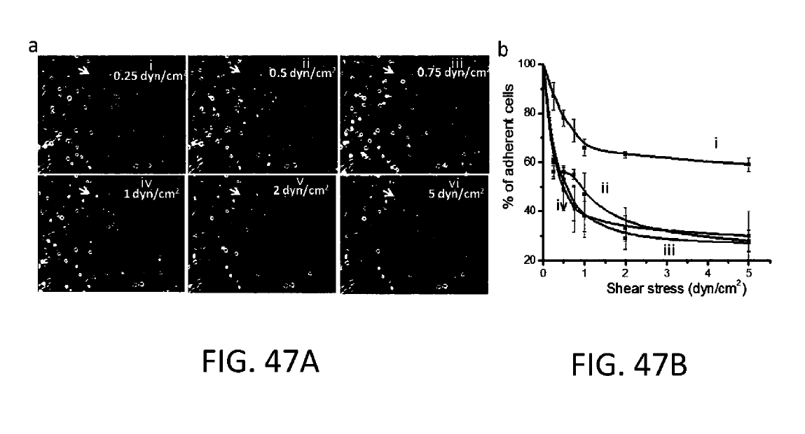

We then studied P-Aptamer-MSC (with controls) and HUVEC interactions using a parallel flow chamber assay. Specifically, a confluent monolayer of HUVECs was first cultured. After histamine treatment, P-Aptamer-MSC were perfused on the endothelium under controlled shear stress in the flow chamber. Significantly, P-Aptamer-MSC bound to HUVEC under static conditions, and accumulated on the HUVEC plate when shear stresses were applied, up to 0.75 dyn/cm² (FIG. 47A). Approximately 60% of MSCs that were initially present before flow conditions remained attached to HUVEC even up to 5 dyn/cm², which is significantly higher than controls including MSC without aptamer modification, scrambled sequence DNA modified MSC, and P-Aptamer-MSC on HUVEC pre-blocked with P-selectin aptamers (FIG. 47B). This strongly suggests that P-selectin binding aptamer conjugation to the MSC surface promoted strong and specific interactions between MSC and HUVEC.

We next studied the L-selectin aptamer promoted cell-cell interactions between MSC and leukocytes (neutrophils). Neutrophils exhibit robust rolling and adhesion on activated endothelium and P-selectin coated surfaces (which resemble activated endothelium). In addition, neutrophils that adhere on activated endothelium further capture free flowing neutrophils via interactions between L-selectin and its ligands (e.g., PSGL-1), which are both expressed on neutrophils. We first validated these native properties of neutrophils using the parallel flow chamber assay and observed robust neutrophil rolling, adhesion, and secondary tethering events on P-selectin coated surfaces, confirming that P-selectin ligands and L-selectin expressed on neutrophils are viable and functional and confirming the reliability of using such an assay to study MSC/neutrophil interactions as described below.

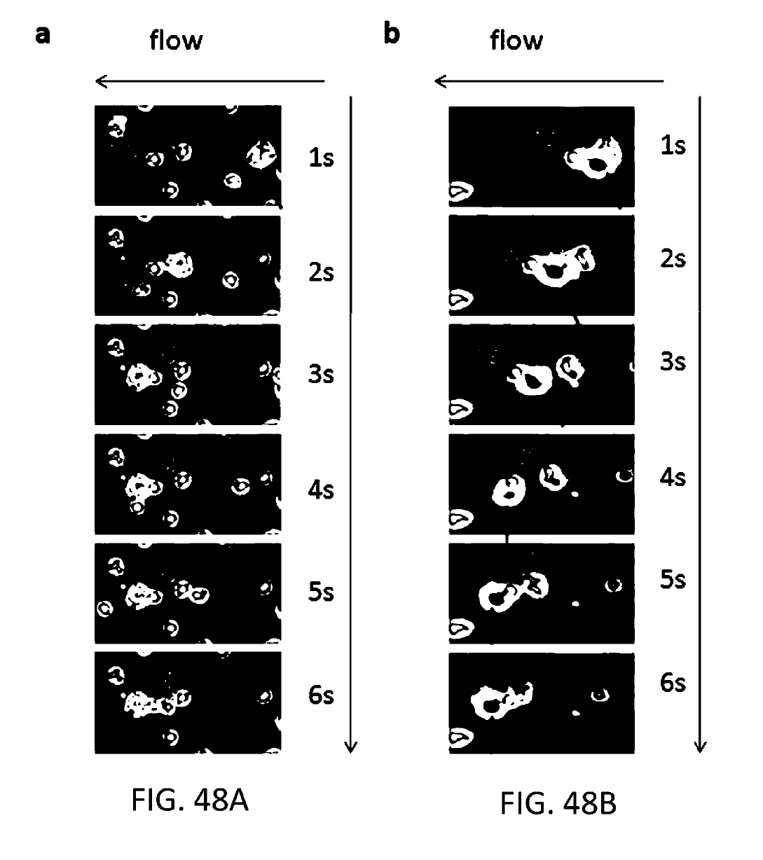

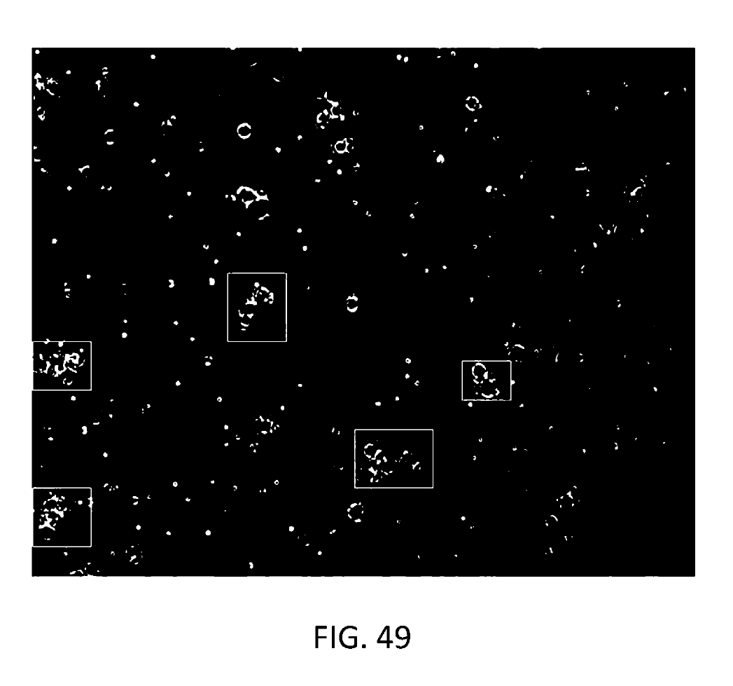

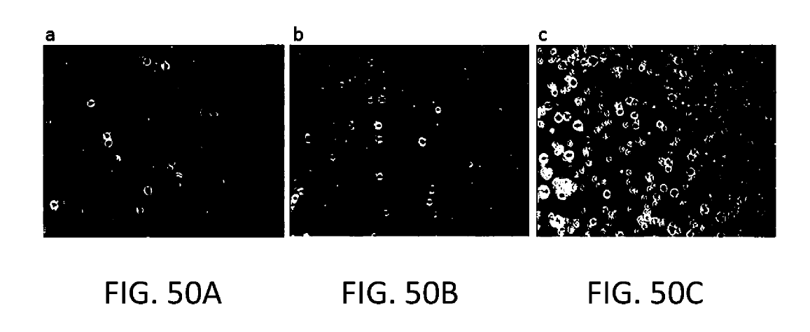

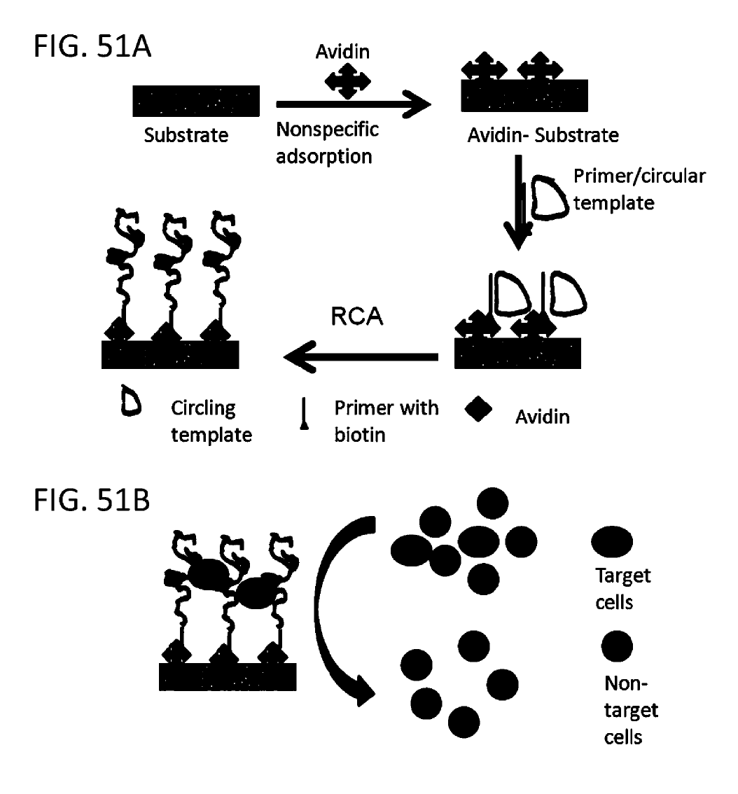

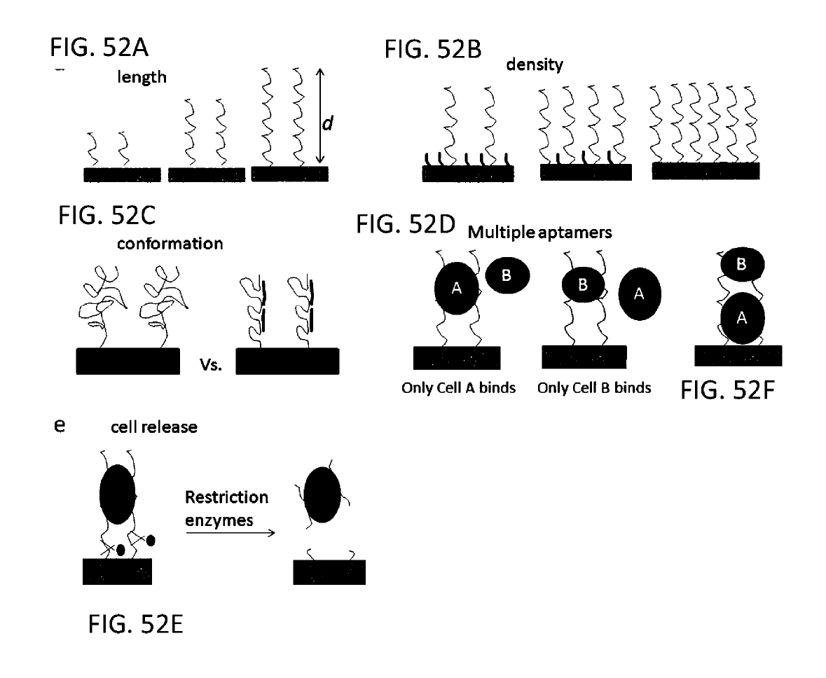

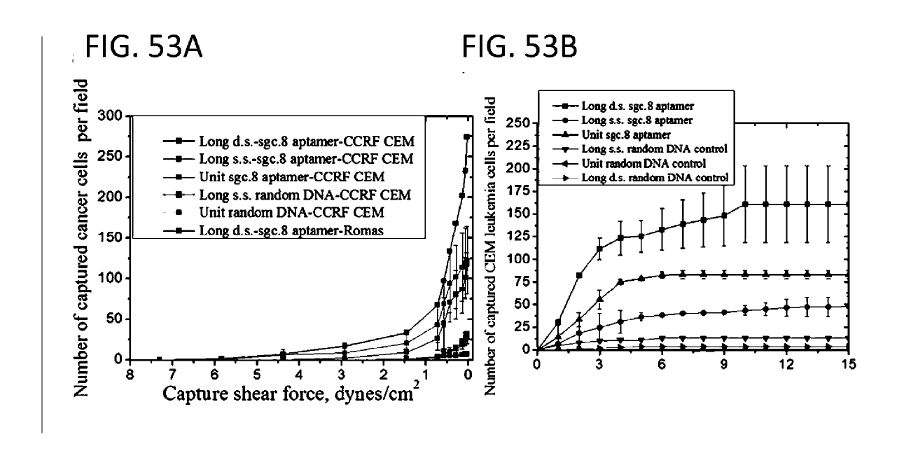

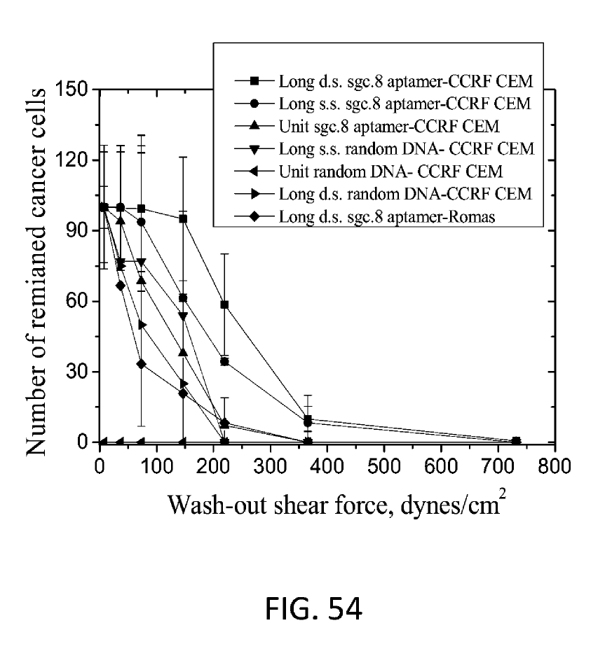

We then investigated the interactions between L-Aptamer-MSC and L-selectin expressing neutrophils. In the flow chamber assay, we first mixed neutrophils (˜2×10⁶) and L-Aptamer-MSCs (˜5×10⁵) and then perfused them immediately over a P-selectin coated surface. Strikingly, we observed that 1) arrested neutrophils on the P-selectin coated surface captured free flowing L-Aptamer-MSC (FIG. 48A, 43B), and 2) neutrophils first complexed with the L-Aptamer-MSCs in the free flowing stream which facilitated tethering of the MSC onto the P-selectin coated surface (FIG. 48B, 43C). Several combinations of MSC/neutrophil complexes were formed in the flow stream: In addition to MSC/neutrophil pairs, MSC were commonly conjugated to two or more neutrophils and in some cases, large multicellular MSC/neutrophil aggregates formed (FIG. 49). Note that these large aggregates could tether to P-selectin coated surfaces under flow conditions through neutrophil/P-selectin interactions. In contrast, for control experiments, minimal MSC/neutrophil interactions or neutrophil-mediated capture of MSC on P-selectin surface were observed where (a) neutrophils and PBS-MSC, (b) neutrophils and scrambled sequence aptamer modified MSC, or (c) neutrophils blocked with L-selectin aptamers and L-Aptamer-MSC were investigated (FIGS. 50A-50C). Interestingly, unlike interactions between L-Aptamer-MSC on L-selectin coated surfaces that were sustained well above 1.5 dyn/cm², L-Aptamer-MSC and neutrophil interactions were only effective under flow conditions at shear stresses of 0.5 dyn/cm² or lower. It is unclear if this is due to 1) cell-cell interactions being ineffective at higher shear stresses and/or 2) the shedding of L-selectins from neutrophil surfaces at higher shear stresses.

### Example 11

### Exemplary Nucleic Acids

Exemplary nucleic acids are shown in the table below.

| SEQ | |
|---|---|
| ID | |
| NO | Sequences (5′->3′) |
| 10 | FAM-AAG GCT ACG GCA CGT AGA GCA TCA CCA TGA TCC TGT GTG GTC TAT GTC |
| | GTC GTT CG |
| 11 | Biotin-CGA ACG ACG ACA TAG ACC ACA-Dabcyl |
| 12 | Cy5-AAG GCT ACG GCA CGT AGA GCA TCA CCA TGA TCC TGT GTG GTC TGT GTC G |
| 13 | Biotin-CGA ACG ACG ACA TAG ACC ACA-Iowa Black RQ |
| 14 | Cy5-AAG GCT ACG GCA CGT AGA GCA TCA CCA TGA TCC TGT GTG GTC TGT GTC G |
| 15 | Biotin-CGA CAC AGA CC/Cy3/A CA |
| 6 | Cy3-TT-Cy5-TTTTTTTT-Biotin |
| 16 | 5′-FAM-tagccaaggtaaccagtacaaggtgctaaacgtaatggcttcggcttac-biotin-3′ |
| 17 | 5′-biotin-tagccaaggtaaccagtacaaggtgctaaacgtaatggcttcggcttac-invert T-3′ |
| 18 | 5′-gatgtagggacagtcaaatggagtggttcaaccgcccatcttcaacaat-biotin-3′ |
| 19 | 5′-gatgtagggacagtcaaatggagtggttcaaccgcccatcttcaacaat-FAM-3′ |
| 20 | 5′-biotin-cucaacgagccaggaacaucgacgucagcaaacgcgag-3′ |
| 21 | 5′-tacgtttagcaccttgtactggttacc-FAM-3′ |

### Other Embodiments

A number of embodiments of the invention have been described. Nevertheless, it will be understood that various modifications may be made without departing from the spirit and scope of the invention. Accordingly, other embodiments are within the scope of the following claims.

1. A composition comprising an isolated cell, wherein a surface of the cell is attached to a nucleic acid that specifically binds to a non-nucleic acid target.

2. The composition of claim 1, wherein the nucleic acid is an aptamer.

3. The composition of claim 1, wherein the nucleic acid is covalently immobilized on the cell surface.

4. The composition of claim 1, comprising a connector moiety between the cell and the nucleic acid

5. The composition of claim 1, wherein the nucleic acid is modified with one or more sensors that enable imaging based detection.

6. The composition of claim 1, wherein nucleic acid comprises two polynucleotide strands.

7. The composition of claim 6, wherein one of the two polynucleotide strands is an aptamer strand and the other one is a complementary strand thereof.

8. The composition of claim 6, wherein both polynucleotide strands are aptamers that can bind to one or more specific target molecules.

9. The composition of claim 1, wherein the nucleic acid binds to a cell surface antigen.

10. The composition of claim 9, wherein the cell surface antigen is a selectin.

11. The composition of claim 1, wherein the non-nucleic acid target is PDGF or thrombin.

12. The composition of claim 1, wherein the nucleic acid binds to the target under physiological conditions.

13. A method comprising contacting the composition of claim 1 with a target and detecting binding of the target to the composition.

14. A method comprising contacting the composition of claim 1 with a cell or surface, such that the composition binds to the cell or surface.

15. A kit comprising the composition of claim 1.

16. A method comprising:
providing a capture agent bound on a solid support;
contacting the capture agent with a solution such that a target of the capture agent binds to the capture agent;
contacting the target bound to the capture agent with a nucleic acid that specifically binds to the non-nucleic acid target, wherein the nucleic acid comprises a primer;
contacting the primer with a circular template at least partially complementary to the primer; and
performing a rolling circle amplification (RCA) reaction using the primer and the template circular template; and
detecting a product of the RCA reaction.

17. The method of claim 16, wherein the capture agent is an apatamer.

18. The method of claim 16, wherein the circular template encodes a catalytic nucleic acid.

19. A composition comprising a sensor moiety immobilized on the surface of a cell, wherein the sensor moiety generates a signal in the presence of a target or condition.

20. The composition of claim 19, wherein the sensor moiety comprises a binding group that specifically binds to the target and a reporter group that generates a signal when the binding group has bound to the target.

21. The composition of claim 19, wherein the sensor moiety comprises a reporter group that generates the signal in the presence of the target or condition.
